(19) United States
(12) Patent Application Publication
(10) Pub. No.: US 2024/0358835 A1
(43) Pub. Date: Oct. 31, 2024

(54) TUMOR INFILTRATING LYMPHOCYTES WITH INCREASED METABOLIC ACTIVITY

(71) Applicant: Orgenesis Inc., Germantown, MD (US)

(72) Inventors: Vered CAPLAN, Tel Aviv (IL); Moran MEIRON, Zichron Yaakov (IL)

(73) Assignee: Orgenesis Inc., Germantown, MD (US)

(21) Appl. No.: 18/683,773

(22) PCT Filed: Aug. 17, 2022
(86) PCT No.: PCT/US2022/040542

§ 371 Date: Feb. 15, 2024

Related U.S. Application Data
(60) Provisional application No. 63/233,854, filed on Aug. 17, 2021.
(60) Provisional application No. 63/252,176, filed on Oct. 5, 2021.

(51) Int. Cl. A61K 39/00; C12N 5/0783

(52) U.S. Cl. CPC A61K 39/464499; A61K 39/4611; A61K 39/464406; A61K 39/464486; A61K 39/464488; C12N 5/0636; C12N 2501/998

(57) ABSTRACT

A population of improved tumor infiltrating lymphocytes (TILs). These TILs have increased metabolic activity (MA), and increased therapeutic efficacy for cancer treatment, allowing applying reduced cell doses to a subject in need thereof. Further disclosed are methods for selecting a population of TILs with increased metabolic activity, methods for producing, and methods for using said TILs.

### FIELD OF DISCLOSURE

Disclosed herein is a population of improved tumor infiltrating lymphocytes (TILs). These TILs have increased metabolic activity (MA), and increased therapeutic efficacy for cancer treatment, allowing applying reduced cell doses to a subject in need thereof. Further disclosed are methods for selecting a population of TILs with increased metabolic activity, methods for producing, and methods for using said TILs.

### BACKGROUND

According to the world health organization, cancer is the second leading cause of death globally and is responsible for 9.6 million deaths in 2018. National expenditures for cancer care in the United States totaled nearly $147.3 billion in 2017. The number of new cancer cases is expected to rise from 14 million to 23.6 million by 2030.

For years, the foundations of cancer treatment were surgery, chemotherapy, and radiation therapy. Over the past several years, immunotherapies have emerged and showed great success. Additionally, there is an emerging family of therapies using cells as the therapeutic agent called adoptive cell therapy (ACT). One of the most successful forms of ACT, which is mostly relevant for metastatic melanoma patients, is based on tumor infiltrating lymphocytes (TILs). TILs are heterogeneous subsets of immune cells which are present within a tumor mass and have the ability to specifically recognize tumor antigens and eliminate malignant cells. This population of cells is characterized by a diversity of phenotypes, antigen specificities, avidities, and functional characteristics, among them anti-tumor reactivity. TILs comprise Tumor Infiltrating Lymphocytes (TILs), as well as other types of infiltrating immune cells. In TIL therapy, TILs are derived and cultured from tumor fragments in a process called pre-REP (rapid expansion protocol) which spans 10-18 days to enable the generation of a TIL culture ready for expansion. The next stage is called REP in which cells are activated, and expanded ex vivo for additional two weeks followed by re-infusion into the tumor-bearing host together with Interleukin-2 (IL-2) as a growth factor. This treatment, following host lympho-depletion, demonstrated objective responsive (OR) rates of 50-70% in stage IV metastatic melanoma patients including some complete tumor regressions. Consequently, this treatment is considered one of the most effective treatments for metastatic melanoma patients to date.

Some of the main drawbacks of this therapy is the time-consuming and labor-intensive manufacturing process, as well as the high cell dosage that is needed for treatment. The therapy is not only limited by its price but mostly limited by scale. As the processing is done manually in highly specialized cancer treatment institutions worldwide with trained and qualified staff, the scale up of the technology and expansion of the market is very limited. The current standard “Young TIL” protocol, relating to minimally cultured TILs with optimal characteristics for adoptive cell therapy, involves the growth and expansion of TILs using 24-well plates, tissue culture flasks and gas-permeable bags, which are not optimal for current good manufacturing practice (cGMP) conditions that are needed for Phase III clinical trials. The vessels used are “open” systems that increase the risk of microbial contamination.

Therefore, there is a dramatic need for innovative technologies that will simplify TILs production, dramatically cut down its cost and allow better scalability and wider availability of the therapy. Similarly, there is a dramatic need for more potent TILs that may reduce the high cell dosage currently required.

### SUMMARY OF THE DISCLOSURE

In one aspect, disclosed herein is a population of tumor infiltrating lymphocytes (TILs) produced by a method comprising: extracting a biopsy comprising a tumor and/or its surroundings; isolating, culturing, and expanding TILs isolated from said biopsy; determining the metabolic activity (MA) in said TILs by a method comprising measuring in the extracellular environment of the cell the time-dependent acidification profiles due to secretion of: (i) non-volatile soluble metabolic products and volatile soluble metabolic products; (ii) non-volatile soluble metabolic products; and (iii) volatile soluble metabolic products; wherein said measuring acidification profile of (ii) is affected in an air-exposed chamber, and wherein said measuring acidification profile of (i) is affected in an air-sealed chamber, and wherein said measuring acidification profile of (iii) is affected by subtracting the acidification profile of (ii) from the acidification profile of (i), and wherein all of said time dependent acidification profiles are indicative of the metabolic activity of the cell; and harvesting TILs.

In some related aspects, TILs are harvested when the MA is above a predetermined threshold.

In some related aspects, the method further comprises contacting expanded TILs isolated from the biopsy with one or more stimulants prior to, or concomitant with determining the metabolic activity. In some related aspects, said one or more stimulants comprise a synthetic peptide. In some related aspects, said one or more stimulants comprise a cancer-specific peptide. In some related aspects, said one or more stimulants are selected from the group consisting of NY-ESO-1, Her-2a, ConA, PHA, MAGE-A3 and glucose.

In some related aspects, said method further comprises prior to harvesting TILs, selecting and expanding TILs having enhanced potency for cell therapy based on said metabolic activity.

In some related aspects, said tumor is selected from melanoma, non-Hodgkin's lymphoma, Hodgkin's disease, leukemia, plasmocytoma, sarcoma, glioma, thymoma, breast cancer, prostate cancer, colorectal cancer, kidney cancer, renal cell carcinoma, uterine cancer, pancreatic cancer, esophageal cancer, brain cancer, lung cancer, ovarian cancer, cervical cancer, testicular cancer, gastric cancer, esophageal cancer, multiple myeloma, hepatoma, acute lymphoblastic leukemia (ALL), acute myelogenous leukemia (AML), chronic myelogenous leukemia (CML), and chronic lymphocytic leukemia (CLL), or any combination thereof.

In some related aspects, said TILs comprise T cells, B cells, natural killer (NK) cells, macrophages, neutrophils, dendritic cells, mast cells, eosinophils, basophils, plasma cells, antigen presenting cells (APCs), CD4+, CD8+, CD163+, CD20+, CD3+, CD138+, CD163+, CD56+, CD28+, CD69+, FoxP3+, DC-LAMP+ cells, or any combination thereof.

In some aspects, disclosed herein is a method for producing a population of tumor infiltrating lymphocytes (TILs), said method comprising: extracting a biopsy comprising a tumor and/or its surroundings; isolating, culturing, and expanding TILs isolated from said biopsy; determining the metabolic activity (MA) in said TILs by a method comprising measuring in the extracellular environment of the cell the time-dependent acidification profiles due to secretion of: (i) non-volatile soluble metabolic products and volatile soluble metabolic products; (ii) non-volatile soluble metabolic products; and (iii) volatile soluble metabolic products; wherein said measuring acidification profile of (ii) is affected in an air-exposed chamber, and wherein said measuring acidification profile of (i) is affected in an air-sealed chamber, and wherein said measuring acidification profile of (iii) is affected by subtracting the acidification profile of (ii) from the acidification profile of (i), and wherein all of said time dependent acidification profiles are indicative of the metabolic activity of the cell; and harvesting TILs.

In some related aspects, TILs are harvested when the MA is above a predetermined threshold.

In some related aspects, the method further comprises contacting expanded TILs isolated from the biopsy with one or more stimulants prior to, or concomitant with determining the metabolic activity. In some related aspects, said one or more stimulants comprise a synthetic peptide. In some related aspects, said one or more stimulants comprise a cancer-specific peptide. In some related aspects, said one or more stimulants are selected from the group consisting of NY-ESO-1, Her-2a, ConA, PHA, MAGE-A3 and glucose.

In some related aspects, said method further comprises prior to harvesting TILs, selecting and expanding TILs having enhanced potency for cell therapy based on said metabolic activity.

In some related aspects, said tumor is selected from melanoma, non-Hodgkin's lymphoma, Hodgkin's disease, leukemia, plasmocytoma, sarcoma, glioma, thymoma, breast cancer, prostate cancer, colorectal cancer, kidney cancer, renal cell carcinoma, uterine cancer, pancreatic cancer, esophageal cancer, brain cancer, lung cancer, ovarian cancer, cervical cancer, testicular cancer, gastric cancer, esophageal cancer, multiple myeloma, hepatoma, acute lymphoblastic leukemia (ALL), acute myelogenous leukemia (AML), chronic myelogenous leukemia (CML), and chronic lymphocytic leukemia (CLL), or any combination thereof.

In some related aspects, said TILs comprise T cells, B cells, natural killer (NK) cells, macrophages, neutrophils, dendritic cells, mast cells, eosinophils, basophils, plasma cells, antigen presenting cells (APCs), CD4+, CD8+, CD163+, CD20+, CD3+, CD138+, CD163+, CD56+, CD28+, CD69+, FoxP3+, DC-LAMP+ cells, or any combination thereof.

In some aspects, disclosed herein is a method for treating a tumor in a subject in need thereof, said treating comprising administering a population of tumor infiltrating lymphocytes (TILs) produced by a method comprising: extracting a biopsy comprising a tumor and/or its surroundings; isolating, culturing, and expanding TILs isolated from said biopsy; determining the metabolic activity (MA) in said TILs by a method comprising measuring in the extracellular environment of the cell the time-dependent acidification profiles due to secretion of: (i) non-volatile soluble metabolic products and volatile soluble metabolic products; (ii) non-volatile soluble metabolic products; and (iii) volatile soluble metabolic products; wherein said measuring acidification profile of (ii) is affected in an air-exposed chamber, and wherein said measuring acidification profile of (i) is affected in an air-sealed chamber, and wherein said measuring acidification profile of (iii) is affected by subtracting the acidification profile of (ii) from the acidification profile of (i), and wherein all of said time dependent acidification profiles are indicative of the metabolic activity of the cell; harvesting TILs; and administering the subject with the TILs. In some related aspects, TILs are harvested when the MA is above a predetermined threshold.

In some related aspects, the method further comprises contacting expanded TILs isolated from the biopsy with one or more stimulants prior to, or concomitant with determining the metabolic activity. In some related aspects, said one or more stimulants comprise a synthetic peptide. In some related aspects, said one or more stimulants comprise a cancer-specific peptide. In some related aspects, said one or more stimulants are selected from the group consisting of NY-ESO-1, Her-2a, ConA, PHA, MAGE-A3 and glucose.

In some related aspects, said method further comprises prior to harvesting TILs, selecting and expanding TILs having enhanced potency for cell therapy based on said metabolic activity.

In some related aspects, said tumor is selected from melanoma, non-Hodgkin's lymphoma, Hodgkin's disease, leukemia, plasmocytoma, sarcoma, glioma, thymoma, breast cancer, prostate cancer, colorectal cancer, kidney cancer, renal cell carcinoma, uterine cancer, pancreatic cancer, esophageal cancer, brain cancer, lung cancer, ovarian cancer, cervical cancer, testicular cancer, gastric cancer, esophageal cancer, multiple myeloma, hepatoma, acute lymphoblastic leukemia (ALL), acute myelogenous leukemia (AML), chronic myelogenous leukemia (CML), and chronic lymphocytic leukemia (CLL), or any combination thereof.

In some related aspects, said TILs comprise T cells, B cells, natural killer (NK) cells, macrophages, neutrophils, dendritic cells, mast cells, eosinophils, basophils, plasma cells, antigen presenting cells (APCs), CD4+, CD8+, CD163+, CD20+, CD3+, CD138+, CD163+, CD56+, CD28+, CD69+, FoxP3+, DC-LAMP+ cells, or any combination thereof.

In some aspects, disclosed herein is a method for monitoring treatment, the method comprising treating a subject in need with TILs; and measuring metabolic activity in TILs of the subject, wherein a shift in the metabolic activity of the TILs towards that of a normal healthy cell sample examined under identical conditions is indicative of an efficacious treatment of the disease.

In some aspects, disclosed herein is a method of measuring the efficacy of therapy, the method comprising treating a subject in need with TILs; and measuring metabolic activity in TILs of the subject, wherein a shift in the metabolic activity of the TILs towards that of a normal healthy cell sample examined under identical conditions is indicative of an efficacious therapy.

In some aspects, disclosed herein is a method for monitoring treatment, the method comprising treating a subject in need with TILs; and measuring metabolic activity in TILs of the subject, wherein a shift in the metabolic activity of the TILs towards an increased glycolysis cycle is indicative of an efficacious treatment of the disease.

In some aspects, disclosed herein is a method of measuring the efficacy of therapy, the method comprising treating a subject in need with TILs; and measuring metabolic activity in TILs of the subject, wherein a shift in the metabolic activity of the TILs towards an increased glycolysis cycle is indicative of an efficacious treatment of the disease.

### BRIEF DESCRIPTION OF THE DRAWINGS

The subject matter disclosed herein is particularly pointed out and distinctly claimed in the concluding portion of the specification. The tumor infiltrating lymphocytes (TILs), method of producing and using thereof, together with objects, features, and advantages thereof, may best be understood by reference to the following detailed description when read with the accompanying drawings in which:

FIG. 1 shows a schematic embodiment of TILs manufacturing method disclosed herein, comprising resecting a tumor, extracting tumor infiltrating lymphocytes (TILs) from the resected tumor, culturing the TILs, measuring the metabolic profile in the cultured TILs, and harvesting TILs comprising an acidification profile above a predetermined threshold. TILS comprising an acidification profile below the threshold can be further cultured or discarded.

FIGS. 2A-2B show growth curves of cells grown in two different runs using T75 flasks with change of media and a bioreactor.

FIG. 3 shows a schematic embodiment of TILs manufacturing method disclosed herein, comprising the pre-REP process which includes the isolation of TILs from the patient's tumor and the establishment of a primary TIL culture having the potential to specifically recognize and eliminate malignant cells, followed by the REP process, where the TILs are further expanded to produce billions of cells that are later re-infused back to the patient.

FIG. 4A shows the number of cells generated from tumor tissues of bladder, breast, lung, kidney, ovarian, uterine, pancreatic and gastrointestinal (GI) cancer. Each point represents the number of cells from a different patient. The average number of cells and standard deviation (SD) are also shown for each type of tumor, where applicable.

FIG. 4B shows flow cytometry analysis of cells generated in pre-REP. CD45+ cells (circles); CD3+CD45+ (squares). The results show high CD45+ leukocyte content, of which the majority are CD3+T cells.

FIG. 5A shows the fold expansion of TILs from various tumors. Fold expansion is calculated as the number of cells counted at the end of REP divided by the number of cells seeded at the start of REP. Each point represents the fold expansion from a different patient. The average fold is shown for each type of tumor, where applicable.

FIG. 5B shows flow cytometry analysis of cells generated in REP. CD45+ cells (circles); CD3+CD45+ (squares).

FIG. 5C shows the T-cell CD4+ and CD8+ subpopulations between pre-REP and REP. Percentage of CD4+ T cells (left) and CD8+ (right) at the completion of pre-REP and REP. Each line connects the points of a different patient at pre-REP and REP.

FIG. 6A shows the REP fold expansion of TILs from three patient's tumors, two with lung cancer and one with gastrointestinal (GI) cancer.

FIG. 6B shows the frequency of T cell subpopulations in the CD45/CD3 population: TN-T naïve, TCM-T central memory, TEM-T effector memory and T-Eff-T effectors. The memory profile was determined by immunostaining for CD45RO/CCR7.

FIG. 6C shows IFNγ secretion from TILs from different donors following stimulation with Dynabeads (CD3/CD28/CD137) for ˜24 hours. IFNγ was determined by ELISA.

FIG. 7A shows the CD4 and CD8 subpopulation frequencies in the TILs from the three patients by flow cytometry analysis. CD8+, CD4+, double positive CD4+CD8+ and double negative CD4-CD8-T Cells.

FIG. 7B shows the flow cytometry analysis of inhibitory and activation markers of the TILs from the three patients, including the inhibitory markers: programmed death receptor 1 (PD-1), T-cell immunoglobulin and mucin domain-containing protein 3 (TIM-3), and lymphocytes activation gene 3 (LAG-3) and the activation marker 4-1BB (CD137).

### DETAILED DESCRIPTION

In the following detailed description, numerous specific details and embodiments are set forth in order to provide a thorough understanding of the population of tumor infiltrating lymphocytes (TILs) disclosed herein, including methods for producing thereof, and methods for treating a tumor in a subject by administering said TILs. In some instances, well-known methods, procedures, and components have not been described in detail so as not to obscure the present disclosure.

### Tumor Infiltrating Lymphocytes (TILs)

In some embodiments, disclosed herein is a population of tumor infiltrating lymphocytes (TILs) produced by a method comprising:
a. extracting a biopsy comprising a tumor and/or its surroundings;
b. isolating, culturing, and expanding TILs isolated from said biopsy;
c. determining the metabolic activity (MA) in said TILs by a method comprising measuring in the extracellular environment of the cell the time-dependent acidification profiles due to secretion of:
   (i) non-volatile soluble metabolic products and volatile soluble metabolic products;
   (ii) non-volatile soluble metabolic products; and
   (iii) volatile soluble metabolic products;
wherein said measuring acidification profile of (ii) is affected in an air-exposed chamber, and
wherein said measuring acidification profile of (i) is affected in an air-sealed chamber, and
wherein said measuring acidification profile of (iii) is affected by subtracting the acidification profile of (ii) from the acidification profile of (i), and
wherein all of said time dependent acidification profiles are indicative of the metabolic activity of the cell; and
d. harvesting TILs.

In some embodiments, TILs are harvested when the MA is above a predetermined threshold.

TILs are white blood cells that leave the bloodstream and migrate towards a tumor and try to attack it. In some embodiments, TILs comprise T cells. In some embodiments, TILs comprise B cells. In some embodiments, TILs comprise natural killer (NK) cells. In some embodiments, TILs comprise macrophages. In some embodiments, TILs comprise neutrophils. In some embodiments, TILs comprise dendritic cells. In some embodiments, TILs comprise mast cells. In some embodiments, TILs comprise eosinophils. In some embodiments, TILs comprise basophils. In some embodiments, TILs comprise plasma cells. In some embodiments, TILs comprise mature dendritic cells. In some embodiments, TILs comprise antigen presenting cells (APCs).

In some embodiments, TILs comprise CD45+ cells. In some embodiments, TILs comprise CD4+ cells. In some embodiments, TILs comprise CD8+ cells. In some embodiments, TILs comprise CD163+ cells. In some embodiments, TILs comprise CD20+ cells. In some embodiments, TILs comprise CD3+ cells. In some embodiments, TILs comprise CD138+ cells. In some embodiments, TILs comprise CD163+ cells. In some embodiments, TILs comprise CD56+ cells. In some embodiments, TILs comprise FoxP3+ cells. In some embodiments, TILs comprise DC-LAMP+ cells. In some embodiments, TILs comprise CD28+ cells. In some embodiments, TILs comprise CD69+ cells.

In some embodiments, TILs comprise a combination of different types of lymphocytes. A skilled artisan would appreciate that different combinations of lymphocytes have different capacities for killing a tumor. In some embodiments, disclosed herein are specific combinations of lymphocytes particularly potent for killing a tumor.

In some embodiments, TILs are modified to express a T cell receptor (TCR) having antigenic specificity for a cancer antigen, e.g., any of the cancer antigens described herein. In some embodiments, a TCR comprises antigenic specificity for a melanoma antigen. In some embodiments, the antigen comprises gp100 or MART-1. In some embodiments, TILs are modified to express a chimeric antigen receptor (CAR) having antigenic specificity for a cancer antigen, e.g., any of the cancer antigens described herein. In some embodiments, TILs are modified to express a cell growth factor that promotes the growth and activation of TILs. In some embodiments, a growth factors comprise T-cell growth factors, IL-2, IL-7, IL-15, or IL-12. In some embodiments, modified TILs express the T-cell growth factor at high levels. T-cell growth factor coding sequences, are readily available in the art, as are promoters, the operable linkage of which to a T-cell growth factor coding sequence promote high-level expression. Suitable methods of modification are known in the art. See, for instance, Sambrook et al., Molecular Cloning: A Laboratory Manual, 3rd ed., Cold Spring Harbor Press, Cold Spring Harbor, N. Y. 2001; and Ausubel et al., Current Protocols in Molecular Biology, Greene Publishing Associates and John Wiley & Sons, NY, 1994. Methods for modifying TILs are known in the art. For example, the TIL may be transduced to express a TCR having antigenic specificity for a cancer antigen using transduction techniques described in Morgan et al., Science 314 (5796): 126-9 (2006) and Johnson et al. Blood 114:535-46 (2009).

Prolonged stimulation of TILs, as occurs during cancer, results in a progressive suppression of the cell's effector function, which is in some embodiments termed “exhaustion”, “senescence” and “anergy”. In some embodiments, TILs exhaustion comprises T cell functional impairment. In some embodiments, TILs exhaustion comprises loss in expression of the effector cytokines interleukin-2 (IL-2), tumor necrosis factor alpha (TNFα), and/or interferon γ (IFNγ). In some embodiments, TILs exhaustion comprises a decrease of cytolytic ability. In some embodiments, TILs exhaustion comprises a decrease in proliferative capacity. In some embodiments, TILs exhaustion comprises sustained upregulated expression of surface inhibitory receptors (IRs). In some embodiments, TILs exhaustion comprises including programmed cell death 1 (PD-1). A skilled artisan would appreciate the term improved TILs refers to the practical reversal and/or to the inhibition of the TILs exhaustion process described above.

In some embodiments, disclosed herein is a population of improved tumor infiltrating lymphocytes (TILs). In some embodiments, improved TILs have increased metabolic activity (MA). In some embodiments, improved TILs have increased therapeutic efficacy for cancer treatment. In some embodiments, improved TILs comprise extended telomeres. In some embodiments, improved TILs telomeres comprise a mean length of at least 0.5 kb. In some embodiments, improved TILs telomeres comprise a mean length of at least 1 kb. In some embodiments, improved TILs telomeres comprise a mean length of at least 2 kb. In some embodiments, improved TILs telomeres comprise a mean length of at least 3 kb. In some embodiments, improved TILs telomeres comprise a mean length of at least 4 kb. In some embodiments, improved TILs telomeres comprise a mean length of at least 5 kb. In some embodiments, improved TILs telomeres comprise a mean length of at least 6 kb. In some embodiments, improved TILs telomeres comprise a mean length of at least 8 kb. In some embodiments, improved TILs telomeres comprise a mean length of at least 9 kb. In some embodiments, improved TILs telomeres comprise a mean length of at least 10 kb. In some embodiments, improved TILs telomeres comprise a mean length of at least 11 kb. In some embodiments, improved TILs telomeres comprise a mean length of at least 12 kb. In some embodiments, improved TILs telomeres comprise a mean length of at least 13 kb. In some embodiments, improved TILs telomeres comprise a mean length of at least 14 kb. In some embodiments, improved TILs telomeres comprise a mean length of at least 15 kb. In some embodiments, improved TILs telomeres comprise a mean length of about 6.3 kb.

In some embodiments, improved TILs telomeres comprise a telomere length of at least 4 kb. In some embodiments, improved TILs telomeres comprise a telomere length of at least 5 kb. In some embodiments, improved TILs telomeres comprise a telomere length of at least 6 kb.

In some embodiments, improved TILs comprise an increased stem cell like phenotype. In some embodiments, improved TILs comprise increased T stem cell memory (TSCM) phenotype. A skilled artisan would appreciate that “T stem cell memory cells” or “TSCM” are a subset of memory lymphocytes endowed with the stem cell-like ability to self-renew and the multipotent capacity to reconstitute the entire spectrum of memory and effector subsets.

In some embodiments, improved TILs comprise increased central memory cell phenotype. A skilled artisan would appreciate that central memory T cells comprise memory T cells localized in secondary lymphoid tissues, which sustain the immune response by proliferating in the secondary lymphoid organs and producing a supply of new effectors.

In some embodiments, improved TILs comprise decreased T effector memory cell phenotype. A skilled artisan would appreciate that effector memory T cells are memory T cells localized in lymphoid and peripheral tissues, which contribute to the immune response by inducing an immediate, but not sustained, defense at pathogen sites of entry.

In some embodiments, improved TILs comprise increased proliferation potential. In some embodiments, improved TILs proliferation potential is increased in at least 10%. In some embodiments, improved TILs proliferation potential is increased in at least 25%. In some embodiments, improved TILs proliferation potential is increased in at least 50%. In some embodiments, improved TILs proliferation potential is increased in at least 75%. In some embodiments, improved TILs proliferation potential is increased in at least 100%. In some embodiments, improved TILs proliferation potential is increased in at least 150%. In some embodiments, improved TILs proliferation potential is increased in at least 200%. In some embodiments, improved TILs proliferation potential is increased in at least 500%. In some embodiments, improved TILs proliferation potential is increased in at least 1000%.

A skilled artisan would appreciate that one assay for measuring proliferation potential may be by clonal growth of a cell in culture until it gets exhausted.

In some embodiments, improved TILs comprise increased anti-tumor activity. A skilled artisan would appreciate that several methods are available in the art to quantify anti-tumor activity, and any of these can be employed for the improved TILs disclosed herein. In some embodiments, improved TILs anti-tumor activity is increased in at least 10%. In some embodiments, improved TILs anti-tumor activity is increased in at least 25%. In some embodiments, improved TILs anti-tumor activity is increased in at least 50%. In some embodiments, improved TILs anti-tumor activity is increased in at least 75%. In some embodiments, improved TILs anti-tumor activity is increased in at least 100%. In some embodiments, improved TILs anti-tumor activity is increased in at least 150%. In some embodiments, improved TILs anti-tumor activity is increased in at least 200%. In some embodiments, improved TILs anti-tumor activity is increased in at least 500%. In some embodiments, improved TILs anti-tumor activity is increased in at least 1000%.

A skilled artisan would appreciate that exhausted TILs and improved TILs can be measured, among others, by the expression of membrane and cell markers, which are well known in the art. Any of these markers can be used to monitor the exhaustion/improvement state of the TILs disclosed herein. In some embodiments, improved TILs comprise increased expression of CCR7. In some embodiments, improved TILs comprise increased expression of CD45RA. In some embodiments, improved TILs comprise increased expression of CD62L. In some embodiments, improved TILs comprise increased expression of CD27. In some embodiments, improved TILs comprise increased expression of CD28. In some embodiments, improved TILs comprise increased expression of CD95. In some embodiments, improved TILs comprise increased expression of IL-2Rbeta. In some embodiments, improved TILs comprise increased expression of IFNγ. In some embodiments, improved TILs comprise increased expression of IL-2. In some embodiments, improved TILs comprise increased expression of ILR7α.

In some embodiments, improved TILs comprise decreased expression of CD45RO. In some embodiments, improved TILs comprise decreased expression of PD-1. In some embodiments, improved TILs comprise decreased expression of LAG-3. In some embodiments, improved TILs comprise decreased expression of TIM-3. In some embodiments, improved TILs comprise decreased expression of TIGIT. In some embodiments, improved TILs comprise decreased expression of FAS ligand. In some embodiments, improved TILs comprise decreased expression of CTLA4.

In some embodiments, improved TILs comprise decreased expression of Sestrin 1. In some embodiments, improved TILs comprise decreased expression of senescence associated beta galactosidase (SA-β-gal).

A skilled artisan would appreciate that besides cell markers, several cellular processes are affected by the exhaustion state of a cell. These processes too can be measured in order to determine the phenotype of the TILs disclosed herein. In some embodiments, improved TILs comprise increased glycolysis. In some embodiments, improved TILs comprise decreased fatty acid oxidation. In some embodiments, improved TILs comprise increased redox potential as measured by NAD+/NADH concentration ratio. In some embodiments, improved TILs comprise increased redox potential as measured by NAD (P)+/NAD (P) H concentration ratio.

In one embodiment, the increased TSCM phenotype comprises increased expression of CD45RA, CCR7, CD95, IL-2Rβ, CD62L, CD27, CD28, IL7Rα or any combination thereof. In another embodiment, the TSCM phenotype comprises increased expression of CD45RA. In another embodiment, the TSCM phenotype comprises increased expression of CCR7. In another embodiment, the TSCM phenotype comprises increased expression of CD95. In another embodiment, the TSCM phenotype comprises increased expression of IL-2Rβ. In another embodiment, the TSCM phenotype comprises increased expression of CD62L. In another embodiment, the TSCM phenotype comprises increased expression of CD27. In another embodiment, the TSCM phenotype comprises increased expression of CD28. In another embodiment, the TSCM phenotype comprises increased expression of IL7Ra. In another embodiment, the TSCM phenotype comprises decreased expression of CD45RO. In another embodiment, the TSCM phenotype comprises increased expression of CD45RA and CCR7. In another embodiment, the TSCM phenotype comprises increased expression of CD45RA and CD95. In another embodiment, the TSCM phenotype comprises increased expression of CD45RA and IL-2Rβ. In another embodiment, the TSCM phenotype comprises increased expression of CD45RA and CD62L. In another embodiment, the TSCM phenotype comprises increased expression of CD45RA and CD27. In another embodiment, the TSCM phenotype comprises increased expression of CD45RA and CD28. In another embodiment, the TSCM phenotype comprises increased expression of CD45RA and IL7Ra. In another embodiment, the TSCM phenotype comprises increased expression of CCR7 and CD95. In another embodiment, the TSCM phenotype comprises increased expression of CCR7 and IL-2Rβ. In another embodiment, the TSCM phenotype comprises increased expression of CCR7 and CD62L. In another embodiment, the TSCM phenotype comprises increased expression of CCR7 and CD27. In another embodiment, the TSCM phenotype comprises increased expression of CCR7 and CD28. In another embodiment, the TSCM phenotype comprises increased expression of CCR7 and IL7Ra. In another embodiment, the TSCM phenotype comprises increased expression of CD95 and IL-2Rβ. In another embodiment, the TSCM phenotype comprises increased expression of CD95 and CD62L. In another embodiment, the TSCM phenotype comprises increased expression of CD95 and CD27. In another embodiment, the TSCM phenotype comprises increased expression of CD95 and CD28. In another embodiment, the TSCM phenotype comprises increased expression of CD95 and IL7Ra. In another embodiment, the TSCM phenotype comprises increased expression of CD62L and CD27. In another embodiment, the TSCM phenotype comprises increased expression of CD62L and CD28. In another embodiment, the TSCM phenotype comprises increased expression of CD62L and IL7Ra. In another embodiment, the TSCM phenotype comprises increased expression of CD27 and CD28. In another embodiment, the TSCM phenotype comprises increased expression of CD27 and IL7Ra. In another embodiment, the TSCM phenotype comprises increased expression of CD28 and IL7Ra. In another embodiment, the TSCM phenotype comprises increased expression of CD45RA, CCR7 and CD95. In another embodiment, the TSCM phenotype comprises increased expression of CD45RA, CCR7 and IL-2Rβ. In another embodiment, the TSCM phenotype comprises increased expression of CCR7, CD95 and IL-2Rβ. In another embodiment, the TSCM phenotype comprises increased expression of CD45RA, CD95 and IL-2Rβ. In another embodiment, the TSCM phenotype comprises decreased expression of CD45RO and increased expression of CD45RA. In another embodiment, the TSCM phenotype comprises decreased expression of CD45RO and increased expression of CCR7. In another embodiment, the TSCM phenotype comprises decreased expression of CD45RO and increased expression of CD95. In another embodiment, the TSCM phenotype comprises decreased expression of CD45RO and increased expression of IL-2Rβ. In another embodiment, the TSCM phenotype comprises decreased expression of CD45RO and increased expression of CD62L. In another embodiment, the TSCM phenotype comprises decreased expression of CD45RO and increased expression of CD27. In another embodiment, the TSCM phenotype comprises decreased expression of CD45RO and increased expression of CD28. In another embodiment, the TSCM phenotype comprises decreased expression of CD45RO and increased expression of IL7Ra.

In one embodiment, a population of primary TILs is improved. In one embodiment, a population of naïve T cells is improved. A skilled artisan would appreciate that naïve T cells are T cells that differentiated in the thymus, and successfully underwent the positive and negative processes of central selection in the thymus. In some embodiments, naïve T cells comprise helper T cells (CD4+), cytotoxic T cells (CD8+), or a combination thereof.

In some embodiments, a population of exhausted TILs is improved. A skilled artisan would appreciate that exhausted T cells comprise distinctive features, as disclosed in the art, as for example high levels of CD43 (1B11), CD69 and inhibitory receptors, but low levels of CD62L and CD127. Further, exhausted T cells are unable to respond to IL-7 and IL-15.

In some embodiments, a population of anergic TILs is improved. A skilled artisan would appreciate that anergic TILs comprise intrinsically inactivated TILs that encountered an antigen but remain alive for an extended period of time in a hyporesponsive state.

In some embodiments, a population of senescent TILs is improved. A skilled artisan would appreciate that senescent TILs comprise specific markers, that can be used to identify them, as for example decreased cell surface receptors CD28 and CD27, and increased expression of CD45RA, CD57, TIGIT and/or KLRG1. Functionally, senescent TILs are also characterized by a poor ability to proliferate due to acquisition of macromolecular damage (e.g., shortened telomeres), upregulation of cyclin-dependent kinase (CDK) inhibitors, and development of senescence associated β-Galactosidase (SA-βGal).

### Preparation of TILs

In some embodiments, disclosed herein is a method for producing a population of tumor infiltrating lymphocytes (TILs), said method comprising:
a. extracting a biopsy comprising a tumor and/or its surroundings;
b. isolating, culturing, and expanding TILs isolated from the biopsy;
c. determining the metabolic activity (MA) in the TILs from step (b); and
d. harvesting TILs from step (c).

In some embodiments, TILs are harvested when the MA is above a predetermined threshold.

In some embodiments, the metabolic activity (MA) is determined by a method comprising measuring, in the extracellular environment of the TILs, the time-dependent acidification profiles due to secretion of:
(i) non-volatile soluble metabolic products and volatile soluble metabolic products;
(ii) non-volatile soluble metabolic products; and
(iii) volatile soluble metabolic products.

In some embodiments, measuring the acidification profile of (ii) is affected in an air-exposed chamber. In some embodiments, measuring the acidification profile of (i) is affected in an air-sealed chamber. In some embodiments, measuring the acidification profile of (iii) is affected by subtracting the acidification profile of (ii) from the acidification profile of (i). In some embodiments, time dependent acidification profiles are indicative of the metabolic activity of the cell.

In some embodiments, disclosed herein is a method for producing a population of tumor infiltrating lymphocytes (TILs), said method comprising:
a. extracting a biopsy comprising a tumor and/or its surroundings;
b. isolating, culturing, and expanding TILs isolated from said biopsy;
c. determining the metabolic activity (MA) in said TILs by a method comprising measuring in the extracellular environment of the cell the time-dependent acidification profiles due to secretion of:
   (i) non-volatile soluble metabolic products and volatile soluble metabolic products;
   (ii) non-volatile soluble metabolic products; and
   (iii) volatile soluble metabolic products;
wherein said measuring acidification profile of (ii) is affected in an air-exposed chamber, and
wherein said measuring acidification profile of (i) is affected in an air-sealed chamber, and
wherein said measuring acidification profile of (iii) is affected by subtracting the acidification profile of (ii) from the acidification profile of (i), and
wherein all of said time dependent acidification profiles are indicative of the metabolic activity of the cell;
d. harvesting TILs.

In some embodiments, TILs are harvested when the level of the phenotype parameter is above, or below, a pre-determined threshold.

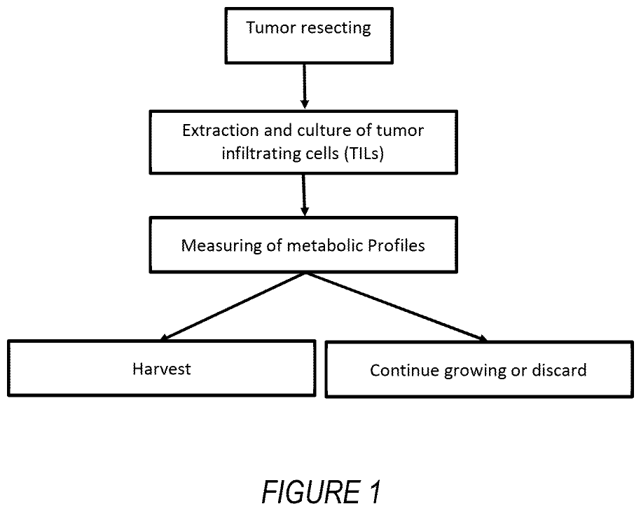

A schematic embodiment of TILs manufacturing method is presented in FIG. 1, which comprises resecting a tumor, extracting tumor infiltrating lymphocytes (TILs) from the resected tumor, culturing the TILs, measuring the MA in cultured TILs, and harvesting TILs comprising MA above a predetermined threshold. TILs comprising MA below the threshold can be further cultured or discarded. In some embodiments, metabolites are measured in the culture medium. In some embodiments, glucose is measured in the culture medium. In some embodiments, lactate is measured in the culture medium. In some embodiments, commercial kits are used to assess the metabolic profile. In one embodiment, the commercial kit is Seahorse XF T Cell Metabolic Profiling Kit (Agilent).

In some embodiments, TILs are extracted tissue. In some embodiments, TILs are extracted from a resected tumor. In some embodiments, TILs are extracted from a non-ablated, non-treated tissue. In some embodiments, TILs are extracted from a biopsy. In some embodiments, the biopsy comprises tumor stroma. In some embodiments, the biopsy comprises a tumor. In some embodiments, the biopsy comprises an ablated tissue. In some embodiments, the biopsy comprises an ablated tumor. In some embodiments, the biopsy comprises a resected tumor. In some embodiments, the biopsy comprises an area surrounding a tumor. In some embodiments, the biopsy comprises necrotic tissue. In some embodiments, the biopsy comprises any combination of a tumor, a tumor stroma, or a tissue surrounding a tumor. In some embodiments, the biopsy comprises tumor tissue devoid of normal tissue and necrotic areas.

In some embodiments, TILs are extracted from a tissue measuring less than 1 mm in each dimension. In some embodiments, TILs are extracted from a tissue measuring about 1 to 2 mm in each dimension. In some embodiments, TILs are extracted from a tissue measuring more than 2 mm in each dimension.

In some embodiments, TILs are extracted from a tissue weighting less than about 0.1 g. In some embodiments, TILs are extracted from a tissue weighting about 0.1 g. In some embodiments, TILs are extracted from a tissue weighting about 0.2 g. In some embodiments, TILs are extracted from a tissue weighting about 0.3 g. In some embodiments, TILs are extracted from a tissue weighting about 0.4 g. In some embodiments, TILs are extracted from a tissue weighting about 0.5 g. In some embodiments, TILs are extracted from a number of single biopsies.

In some embodiments, a biopsy is homogenized by any known tissue homogenization method. In some embodiments, a biopsy is homogenized by fragmentation. In some embodiments, a biopsy is homogenized by fine needle aspiration. In some embodiments, a biopsy is homogenized by enzymatic digestion.

In some embodiments, TILs production comprises a “pre-Rapid Expansion Protocol (pre-REP)”, and a “Rapid Expansion Protocol (REP)” step. In some embodiments, the pre-REP step, comprises a first expansion of TILs in standard lab media such as RPMI comprising reagents such as irradiated feeder cells and anti-CD3 antibodies. The pre-REP continues until a population of about 50×10⁶ cells is reached. In some embodiments, these cells are called herein “young” TILs. In the REP step, the population of young-TILs is further expanded until a cell number relevant for clinical use is obtained.

In some embodiments, biopsy tissues of 1-2 mm are plated in a cell culture plate after homogenization. In some embodiments, for the pre-REP stage biopsy tissues are plated in a 6-well plate, a 12-well plate, a 24-well plate, a 48-well plate, or a 96-well plate. In some embodiments, for the pre-REP stage biopsy tissues are plated in a bioreactor. In some embodiments, biopsies are plated in a medium. In some embodiments, the medium is RMPI 1640 medium containing 10% human serum, 25 mmol/l HEPES pH 7.2, 100 U/ml penicillin, 100 μg/ml streptomycin, 50 μg/ml gentamycin, 5.5×10⁻⁵ mol/l 2-mercaptoethanol, and 3000 IU/ml interleukin. In some embodiments, the medium is PRIME-XV T cell chemically defined media (CDM) supplemented with 3,000 IU/ml interleukin and 10 μg/mL gentamicin. In some embodiments, the medium can be replaced, for example about 1 week after culture initiation.

In some embodiments, the interleukin comprises IL-2. In some embodiments, the interleukin comprises IL-7. In some embodiments, the interleukin comprises IL-15. In some embodiments, the interleukin comprises IL-21.

In some embodiments, the interleukin comprises IL-2, IL-7, IL-15, IL-21, or any combination thereof.

In some embodiments, IL-2 is further supplemented to the media. In some embodiments, IL-2 is supplemented on days 5, 9 and 12. In some embodiments, IL-2 is supplemented on day 5. In some embodiments, IL-2 is supplemented on day 9. In some embodiments, IL-2 is supplemented on day 12.

TILs grow to form a carpet surrounding the tumor cells. In some embodiments, TILs surround the tumor in about 4-7 days, about 7-14 days, or about 14-21 days. In some embodiments, cells are grown and maintained at a concentration lower than about 0.1×10⁶ cells/ml concentration during the pre-REP stage. In some embodiments, cells are grown and maintained at a concentration of about 0.1-0.5×10⁶ cells/ml in the pre-REP stage. In some embodiments, cells are grown and maintained at a concentration of about 0.5-1×10⁶ cells/ml in the pre-REP stage. In some embodiments, cells are grown and maintained at a concentration of about 1-1.5×10⁶ cells/ml in the pre-REP stage. In some embodiments, cells are grown and maintained at a concentration of about 1.5-2×10⁶ cells/ml in the pre-REP stage. In some embodiments, cells are grown and maintained at a concentration of about 2-3×10⁶ cells/ml in the pre-REP stage. In some embodiments, cells are grown and maintained at a concentration of about 3-5×10⁶ cells/ml in the pre-REP stage. In some embodiments, cells are grown and maintained at a concentration higher than about 0.1-0.5×10⁶ cells/ml in the pre-REP stage. When cells are confluent, the contents are mixed and split into daughter wells.

In some embodiments, the pre-REP stage terminates when a number of less than about 10×10⁶ cells are obtained. In some embodiments, the pre-REP stage terminates when a number of about 10×10⁶ cells are obtained. In some embodiments, the pre-REP stage terminates when a number of about 25×10⁶ cells are obtained. In some embodiments, the pre-REP stage terminates when a number of about 50×10⁶ cells are obtained. In some embodiments, the pre-REP stage terminates when a number of about 75×10⁶ cells are obtained. In some embodiments, the pre-REP stage terminates when a number of about 100×10⁶ cells are obtained. In some embodiments, the pre-REP stage terminates when a number of more than 100×10⁶ cells are obtained.

In some embodiments, the pre-REP stage terminates when the lactate concentration in the medium is in the range of 1-5 mM. In some embodiments, the pre-REP stage terminates when the lactate concentration in the medium is 1 mM. In some embodiments, the pre-REP stage terminates when the lactate concentration in the medium is 2 mM. In some embodiments, the pre-REP stage terminates when the lactate concentration in the medium is 3 mM. In some embodiments, the pre-REP stage terminates when the lactate concentration in the medium is 4 mM. In some embodiments, the pre-REP stage terminates when the lactate concentration in the medium is 5 mM.

In some embodiments, TILs are cryopreserved and thawed during their expansion. In some embodiments, TILs are cryopreserved after finishing the pre-REP stage, and are thawed before starting the REP stage. In some embodiments, the TILs are immediately seeded into the REP stage without cryopreservation.

In some embodiments, any suitable culture medium can be used to grow TILs during REP stage. In some embodiments, TILs are thawed two days before the REP stage and suspended in a TIL culture medium (TIL-CM). In some embodiments, a TIL-CM comprises 10% fetal bovine serum, 25 mmol/l HEPES pH 7.2, 100 U/ml penicillin, 100 μg/ml streptomycin, and 2 mmol L-glutamine, 5.5×10−5 mol/l 2-mercaptoethanol in RPMI 1640. In some embodiments, TILs are then seeded in culture plates and expanded. In some embodiments, cells are cultured in AIM-V cell medium, comprising L-glutamine, 50 μg/ml streptomycin sulfate, and 10 μg/ml gentamicin sulfate. In some embodiments TILs are seeded in PRIME XV CDM supplemented with 30 ng/mL anti-CD3 antibody (OKT-3) and 3,000 IU/mL IL-2.

In some embodiments, expanding the number of TILs may comprise using about 2,000 mL to about 12,000 mL of cell medium. In some embodiments, expanding the number of TIL may comprise using about 4,000 mL to about 11,000 mL of cell medium. In some embodiments, expanding the number of TIL may comprise using about 5,000 mL to about 10,000 mL of cell medium. In some embodiments, expanding the number of TIL may comprise using about 6,000 mL to about 9,000 mL of cell medium.

In some embodiments, culture plates for the REP stage are selected from a 6-well plate, a 12-well plate, a 24-well plate, a 48-well plate, or a 96-well plate. In some embodiments, cells are seeded in a bioreactor for the REP stage. In some embodiments, TILs are seeded at a concentration lower than about 0.1×10⁶ cells per ml in the REP stage. In some embodiments, TILs are seeded at a concentration between about 0.1-0.5×10⁶ cells per ml in the REP stage. In some embodiments, TILs are seeded at a concentration between about 0.5-1×10⁶ cells per ml in the REP stage. In some embodiments, TILs are seeded at a concentration between about 1-2.5×10⁶ cells per ml in the REP stage. In some embodiments, TILs are seeded at a concentration between about 2.5-5×10⁶ cells per ml in the REP stage. In some embodiments, TILs are seeded at a concentration higher than about 5×10⁶ cells per ml in the REP stage. In some embodiments, TILs are seeded at a concentration between about 8×10³-16×10³ cells per ml in the REP stage.

In some embodiments, TILs are expanded after seeding in the REP stage. In some embodiments, TILs are expanded by about 50-fold during REP stage. In some embodiments, TILs are expanded by about 60-fold during REP stage. In some embodiments, TILs are expanded by about 70-fold during REP stage. In some embodiments, TILs are expanded by about 80-fold during REP stage. In some embodiments, TILs are expanded by about 90-fold during REP stage. In some embodiments, TILs are expanded by about 100-fold during REP stage.

In some embodiments, TILs are expanded by about 200-fold during REP stage. In some embodiments, TILs are expanded by about 300-fold during REP stage. In some embodiments, TILs are expanded by about 400-fold during REP stage. In some embodiments, TILs are expanded by about 500-fold during REP stage. In some embodiments, TILs are expanded by about 600-fold during REP stage. In some embodiments, TILs are expanded by about 700-fold during REP stage. In some embodiments, TILs are expanded by about 800-fold during REP stage. In some embodiments, TILs are expanded by about 900-fold during REP stage.

In some embodiments, TILs are expanded by about 1000-fold during REP stage. In some embodiments, TILs are expanded by about 1500-fold during REP stage. In some embodiments, TILs are expanded by about 2000-fold during REP stage. In some embodiments, TILs are expanded by more than about 2000-fold during REP stage.

In some embodiments, TILs are expanded by stimulating them with an antigen from a cancer cell. In some embodiments, TILs are expanded by stimulating them with human leukocyte antigen A2 (HLA-A2) binding peptide. In some embodiments, TILs are expanded by stimulating them with MART-1:26-35 peptide. In some embodiments, TILs are expanded by stimulating them with gp100: 209-217 peptide. In some embodiments, TILs are expanded by stimulating them with NY-ESO-1. In some embodiments, TILs are expanded by stimulating them with TRP-1. In some embodiments, TILs are expanded by stimulating them with TRP-2. In some embodiments, TILs are expanded by stimulating them with tyrosinase cancer antigen. In some embodiments, TILs are expanded by stimulating them with MAGE-A3. In some embodiments, TILs are expanded by stimulating them with SSX-2. In some embodiments, TILs are expanded by stimulating them with VEGFR2. In some embodiments, TILs are expanded by stimulating them with a portion of an antigen. In some embodiment, a TIL stimulating antigen is expressed from a vector.

In some embodiments, TILs are expanded by providing them with a T-cell growth factor. In some embodiments, TILs are expanded by providing them with IL-2. In some embodiments, TILs are expanded by providing them with IL-15. In some embodiments, IL-2 is provided in a concentration of about 3000 IU/mL. In some embodiments, IL-2 is provided in a concentration of about 6000 IU/mL.

In some embodiments, TILs are expanded by providing them with an anti-CD3 antibody. In some embodiments, an anti-CD3 antibody is provided in a concentration of about 30 ng/mL.

In some embodiments, TILs are expanded by providing them with irradiated feeder cells. In some embodiments, feeder cells comprise peripheral blood mononuclear cells (PBMC). In some embodiments, feeder cells comprise irradiated allogeneic feeder cells. In some embodiments, feeder cells comprise irradiated autologous feeder cells. In some embodiments, feeder cells comprise artificial antigen presenting cells. In some embodiments, feeder cells comprise K562 leukemia cells transduced with nucleic acids encoding CD3 and/or CD8.

In some embodiments, irradiated feeder cells are provided at a ratio of about 20:1 feeder cells to TILs. In some embodiments, irradiated feeder cells are provided at a ratio of about 25:1 feeder cells to TILs. In some embodiments, irradiated feeder cells are provided at a ratio of about 50:1 feeder cells to TILs. In some embodiments, irradiated feeder cells are provided at a ratio of about 100:1 feeder cells to TILs. In some embodiments, irradiated feeder cells are provided at a ratio of about 200:1 feeder cells to TILs.

In some embodiments, irradiated feeder cells are provided in a ratio from about 1 to about 20 feeder cells to TILs. In some embodiments, irradiated feeder cells are provided in a ratio from about 20 to about 50 feeder cells to TILs. In some embodiments, irradiated feeder cells are provided in a ratio from about 50 to about 100 feeder cells to TILs. In some embodiments, irradiated feeder cells are provided in a ratio from about 100 to about 200 feeder cells to TILs.

In some embodiments, TILs are grown and maintained at a concentration lower than about 0.1×10⁶ cells/ml in the REP stage. In some embodiments, TILs are grown and maintained at a concentration of about 0.1-0.5×10⁶ cells/ml in the REP stage. In some embodiments, TILs are grown and maintained at a concentration of about 0.5-1×10⁶ cells/ml in the REP stage. In some embodiments, TILs are grown and maintained at a concentration of about 1-2.5×10⁶ cells/ml in the REP stage. In some embodiments, TILs are grown and maintained at a concentration of about 2.5-5×10⁶ cells/ml in the REP stage. In some embodiments, TILs are grown and maintained at a concentration higher than about 5×10⁶ cells/ml in the REP stage. In some embodiments, TILs are grown and maintained at a concentration of about 1×10⁶ cells/ml in the REP stage. When cells are confluent, the contents are split into daughter wells.

In some embodiments, TILs are expanded for about 7 days during the REP stage. In some embodiments, TILs are expanded for about 14 days during the REP stage. In some embodiments, TILs are expanded for about 21 days during the REP stage. In some embodiments, TILs are expanded for less than 7 days during the REP stage. In some embodiments, TILs are expanded for between about 7-14 days during the REP stage. In some embodiments, TILs are expanded for between about 14-21 days during the REP stage. In some embodiments, TILs are expanded for more than 21 days during the REP stage.

In some embodiments, the REP stage is terminated when lactate concentration in the medium is in the range of 1-5 mM. In some embodiments, the REP stage is terminated when lactate concentration in the medium is 1 mM. In some embodiments, the REP stage is terminated when lactate concentration in the medium is 2 mM. In some embodiments, the REP stage is terminated when lactate concentration in the medium is 3 mM. In some embodiments, the REP stage is terminated when lactate concentration in the medium is 4 mM. In some embodiments, the REP stage is terminated when lactate concentration in the medium is 5 mM.

In some embodiments, the anti-tumor activity of TILs is monitored, allowing to select TIL populations particularly efficient for lysing cancer cells. In some embodiments, TILs capable of lysing cancer cells may be selected by identifying TILs having any suitable trait associated with the lysis of cancer cells. In some embodiments, TILs are selected according to their IFN-γ release upon co-culture with autologous tumor cells. In some embodiments, particularly efficient TILs release about 50 pg/ml or more of IFN-γ upon co-culture with tumor cells. In some embodiments, particularly efficient TILs release about 100 pg/ml or more of IFN-γ upon co-culture with tumor cells. In some embodiments, particularly efficient TILs release about 200 pg/ml or more of IFN-γ upon co-culture with tumor cells. In some embodiments, particularly efficient TILs release about 300 pg/ml or more of IFN-γ upon co-culture with tumor cells.

In some embodiments, particularly efficient TILs are selected or isolated according to cell surface expression. In some embodiments, TILs are selected according to CD8 expression. In some embodiments, TILs are selected according to CD27 expression. In some embodiments, TILs are selected according to CD28 expression. In some embodiments, TILs are selected according to telomere length. Without being bound to a particular theory, it is believed that cell surface expression of one or more of CD8, CD27, and CD28 and longer telomere lengths are associated with positive objective clinical responses in patients and persistence of the cells in vivo.

In some embodiments, the anti-tumor activity of TILs is predicted by the metabolic activity (MA) of the cells. In some embodiments, the anti-tumor activity of TILs is predicted by the aerobic glycolysis of the cells. In some embodiments, the anti-tumor activity of TILs is predicted by lactate levels in the cells. In some embodiments, the anti-tumor activity of TILs is predicted by pyruvate mitochondrial levels. In some embodiments, the anti-tumor activity of TILs is predicted by acetyl-CoA mitochondrial or cytosolic levels. In some embodiments, the anti-tumor activity of TILs is predicted by aspartate levels.

In some embodiments, the anti-tumor activity of TILs is predicted by nutrient transporter expression and/or activation of the key metabolic regulator mTOR. In some embodiments, the anti-tumor activity of TILs is predicted by PI3K activity. In some embodiments, the anti-tumor activity of TILs is predicted by Akt phosphorylation. In some embodiments, the anti-tumor activity of TILs is predicted by glycolytic enzyme hexokinase II phosphorylation. In some embodiments, the anti-tumor activity of TILs is predicted by 4E-BP1 and/or p70S6 kinase phosphorylation. In some embodiments, the anti-tumor activity of TILs is predicted by sterol regulatory element-binding protein 2 (SREBP2) activation.

In some embodiments, the anti-tumor activity of TILs is predicted by up-regulation of transcription factors c-Myc, estrogen-related receptor α (ERRα), and hypoxia inducible factor-1α (HIF-1α). In some embodiments, the anti-tumor activity of TILs is predicted by AP4 expression.

In some embodiments, the method for producing a population of TILs disclosed herein further comprises contacting TILs with one or more stimulants. In some embodiments, the method for producing a population of TILs disclosed herein further comprises contacting TILs with one or more stimulants, and selecting and expanding TILs having enhanced potency for cell therapy based on the metabolic activity. In some embodiments, the method for producing a population of TILs disclosed herein further comprises selecting and expanding TILs having enhanced potency for cell therapy based on the metabolic activity. In some embodiments, the method for producing a population of TILs disclosed herein further comprises selecting and expanding TILs having increased metabolic activity. In some embodiments, selecting and expanding TILs having increased metabolic activity occurs prior to harvesting TILs. In some embodiments, TILs having enhanced potency for cell therapy have increased metabolic activity (MA). In some embodiments, contacting TILs with one or more stimulants occurs prior to, or concomitant with determining the metabolic activity. In some embodiments, contacting TILs with one or more stimulants occurs prior to determining the metabolic activity. In some embodiments, contacting TILs with one or more stimulants occurs concomitant with determining the metabolic activity.

In some embodiments, the TILs are cultured and expanded in a bioreactor. As skilled artisan would understand the term “bioreactor” any manufactured device or system that supports a biologically active environment. In some embodiments, a bioreactor is a vessel in which a process is carried out which involves organisms or biochemically active substances derived from such organisms.

In some embodiments, any factor beneficial for the culture and growth of TILs described herein may be added to the liquid or culture medium. In some embodiments, a factor dissolves within the liquid, wherein the liquid represents a solvent and the factor a solute to form a solution. In some embodiments, a factor remains as a particulate within the liquid. In some embodiments, a liquid or a culture medium comprises a standard lab media used for growing TILs such as RPMI. In some embodiments, a liquid or culture medium comprises reagents such as irradiated feeder cells. In some embodiments, a liquid or culture medium comprises a TIL culture medium (TIL-CM). In some embodiments, a liquid or culture medium comprises fetal bovine serum, HEPES pH 7.2, penicillin, streptomycin, L-glutamine, 2-mercaptoethanol, or any combination thereof. In some embodiments, a liquid or culture medium comprises RPMI 1640 medium. In some embodiments, a liquid or culture medium comprises AIM-V cell medium.

In some embodiments, the above-mentioned methods use a sensor suitably connected to a controller for monitoring TILs metabolic activity (MA). In some embodiments, the sensor allows performing a colorimetric assay. In some embodiments, MA is measured by an ELISA reader. In some embodiments, NAD+/NADH ratio is measured by any method known in the art, as for example in-vivo fluorimeter reflectometer, or other luminescent, and/or enzymatic methods.

### Metabolic Activity (MA)

In some embodiments, the methods disclosed herein comprise identifying and selecting TILs with increased metabolic activity (MA). In some embodiments, TILs with increased MA comprise higher efficiency in inhibiting tumors. A method for identifying cells with increased MA is disclosed in U.S. Pat. No. 8,728,758, which is incorporated herein by reference, and some aspects of it are summarized below. This method measures the rate of the metabolic activity of microliter cellular samples, by monitoring extracellular acidification using a pH-sensitive impermeable fluorescence probe.

An artisan would appreciate that cells having “increased metabolic activity” or “improved metabolic activity” may encompass a higher a metabolic activity, as measured, for example, by the methods described herein in detail, than the metabolic activity of untreated cells or cells of a normal healthy cell sample. In some embodiments, increased metabolic activity comprises metabolic activity showing an equivalent or increased glycolysis cycle, as compared with untreated cells or cells of a normal healthy cell sample. In some embodiments, increased metabolic activity comprises metabolic activity showing an equivalent or reduced oxidative phosphorylation cycle, as compared with untreated cells or cells of a normal healthy cell sample. In some embodiments, the metabolic activity is increased by at least 10%. In some embodiments, the metabolic activity is increased by at least 25%. In some embodiments, the metabolic activity is increased by at least 50%. In some embodiments, the metabolic activity is increased by at least 75%. In some embodiments, the metabolic activity is increased by at least 100%. In some embodiments, the metabolic activity is increased by at least 150%. In some embodiments, the metabolic activity is increased by at least 200%. In some embodiments, the metabolic activity is increased by at least 500%. In some embodiments, the metabolic activity is increased by at least 1000%.

As disclosed herein, MA can be used as a tool, for identifying TILs with increased anti-tumor activity. The MA comprises measuring metabolic product profiles in “open” versus “close” (air-sealed) wells. Both records enable to measure the accumulations of soluble versus volatile metabolic products (lactic acid versus CO2 and NH3), thereby differentiating between three metabolic pathways—oxidative phosphorylation, anaerobic glycolysis and aerobic glycolysis, as interpreted below.

Non activated T cells (naive T cells), like most normal differentiated cells, rely primarily on mitochondrial oxidative phosphorylation to efficiently generate ATP for the energy needed for cellular processes, and the volatile CO2 product. In the absence of oxygen they must rely on much less efficient metabolic pathway of ATP production, associated with lactic acid production known as anaerobic glycolysis. In contrast, most cancer cells are found to rely on aerobic glycolysis, which is similar to the anaerobic glycolysis despite the presence of oxygen (“the Warburg effect”). The “Warburg effect” has been shown to occur also in fresh PBMCs of cancer patients. Without being bound by theory, the immunometabolic rational of the “Warburg effect”, namely the shift between naive and activated lymphocytes in fresh PBMCs of cancer patients, may be related to the need of aggressive and effective physiological function of activated T cells in the tumor cells neighborhood, where at early stages before angiogenesis it is probably oxygen deficient. This idea is consistent with the fact that tumor cells are initially adapted to oxygen deficiency through the “Warburg effect”. In light of the above metabolic pathways, the end products, CO2 and lactate contribute directly to the acidification examined by the MA test.

Moreover, another end product which is considered to play a major role in the MA test is Ammonia (NH₃). One of the primary sources of cell energy is protein catabolism which is the process of protein brake down to amino acids. Amino groups are removed from amino acids and converted to ammonia. Another source of cellular NH3 production is through metabolic pathways of purines and pyrimidines making up the two groups of nitrogenous bases. In the present measurement system, as in vivo, vital cells must maintain the cytoplasm in a constant pH of about 7.2-7.4 by secretion of the metabolic acidic and basic products, such as lactic acid, carbonic acids and the ammonium base.

In some embodiments, provided herein is a method of measuring a metabolic activity (MA) of TILs, the method comprising measuring in an extracellular environment of the cell, time-dependent acidification profiles due to secretion of:
(i) non-volatile soluble metabolic products;
(ii) non-volatile soluble metabolic products and volatile soluble metabolic products;
(iii) volatile soluble metabolic products;
wherein at least one of the time dependent acidification profiles is indicative of the metabolic activity pathway of the TILs.

As used herein “metabolic activity pathway” refers to the relative contribution of mitochondrial oxidative phosphorylation, anaerobic glycolysis, aerobic glycolysis and NH3+production to energy production.

As used herein “independently measuring” refers to separate measuring of items (i), (ii) and possibly (iii). Although it will be appreciated, according to a specific embodiment, that (iii) is the result of subtracting (i) from (ii). These separate measurements can be performed in parallel, simultaneously, on identical yet separate cell samples, or sequentially on a single cell sample.

Measurement of metabolic activity is performed by calculating the accumulated acidification in relation to the fluorescencently measured pH changes in the extracellular environment of the cells (e.g., pmol/ul/hour/2500 cells) in “open” and “close” state. It will be appreciated that, according to a specific embodiment, this measurement is performed only in the extracellular environment of the cell and not intracellularly. Extracellular pH measurement is advantageous since in the extracellular environment there is a persistent acidic accumulation versus a relatively small average changes in the transient intracellular responses due to homeostatic physiological regulation; there is no physiological interference of the extracellular probe with intracellular processes; there is a comparative high signal to noise ratio of the extracellular ratiometric fluorescent probe; simplicity of fluorescent medium (calibrated buffer capacity) preparation versus cellular manipulations; there is no background fluorescence in contrast to significant leakage of intracellular probes; kinetic measurements are made with no need for permeabilization procedures, thereby allowing the analysis of live cells in real-time; there are minimal problems associated with quenching and oxidation effects; and finally simultaneous high throughput kinetic measurements are enabled without the above hurdles.

As used herein “an extracellular environment” of the cell refers to a natural environment e.g., blood or plasma, or an artificial environment such as a culture medium. According to a specific embodiment, the MA test is affected in a defined solution (all components are known) having a calibrated buffered capacity. It will be appreciated that the buffer capacity should ensure minor changes in the physiological pH.

According to a specific embodiment, the buffer is a phosphate buffer (e.g., phosphate buffer saline 1-10 mM or 10 mM phosphate buffer). It will be appreciated that low buffer concentration is required for acidification measurements at low cell concentration. According to a specific embodiment 10 mM phosphate buffer saline is used for 2.5×10⁶ cells/ml.

In some embodiments, kinetics of metabolic activity is monitored during the incubation by a minor acidification process of a HPTS fluorescence calibrated buffer capacity. According to a specific embodiment, measuring the acidification profiles is performed at a constant temperature, e.g., 20-40° C. or specifically, at optimal growth temperature, say 37° C. for mammalian cells.

As described herein, the extracellular acidification profiles are indicative of the identity of the various metabolic products secreted by the cell.

A skilled artisan would appreciate that activated T cells use preferentially aerobic glycolysis which is characterized mainly by the secretion of Lactate to the medium. In contrast, a differentiated tissue employs oxidative phosphorylation or anaerobic glycolysis and therefore will secrete CO₂ or lactate, dependent on the availability of oxygen, respectively.

According to a specific embodiment, time dependent acidification profile due to secretion of non-volatile soluble metabolic products mainly lactate is performed in an air-exposed chamber. Under such conditions (“open”), there is gas ventilation of CO₂ and NH₃, so that only lactate acid production (including other non-volatile organic acids) contributes to the equivalent acidic accumulation in each well.

According to a specific embodiment, time dependent acidification profile due to secretion of non-volatile soluble metabolic products and volatile soluble metabolic products are affected in an air-sealed chamber. In the hermetically sealed state (“close”), CO₂ and NH₃ react at equilibrium with water to form carbonic acid and basic ammonium ions. In this state, however, the NH4+basic cation titrates the acidity level produced by both the lactic and carbonic acid anions around pH 7.

According to a specific embodiment, the acidification kinetics is measured in 30 minutes sequence of air “open” and “closed” states of the multi well plate.

By the appropriate rates (V), of acidification (+) and basic titration (−), the total measured rates of acidification in the open state (Vopen) and the closed state (Vclosed) are described by the coupled equations:

$$Vopen = V ( lactic acid ) . \\ Vclose = V ( lactic acid ) + V ( carbonic acid ) - V ( ammonium base ) .$$

Using this configuration, the time-dependent acidification profile due to secretion of volatile soluble metabolic products is calculated by the subtraction of the profiles of (ii)-(i).

In some embodiments, measuring the kinetics of extracellular acidification is performed using a non-toxic membrane impermeable probe. Examples include, but are not limited to, a ratiometric pH probe, a CO₂ probe, an NH₃ probe, a lactate probe and a combination of same. According to a specific embodiment the ratiometric technique is required for the high sensitivity at pH buffered conditions.

Examples of specific probes which can be used according to the present teachings include, but are not limited to, HPTS, CFDA and carboxy fluorescein.

According to a specific embodiment, measuring the acidification is affected using the ratiometric pH probe 8-Hydroxypyrene-1,3,6-trisulfonic acid (HPTS). HPTS is a cost effective, non-toxic, highly water-soluble membrane-impermeant pH indicator with a pKa of ^{˜}7.3 in aqueous buffers. HPTS exhibits a pH-dependent absorption shift, allowing ratiometric pH measurements as function of the ratio between the fluorescence intensities at 513 nm that are measured sequentially under excitation at 455 nm and 403 nm. This method is essential for the present sensitive measurements of minor pH changes in the physiological range around pH 7.

According to a specific embodiment, the fluorescent probe is attached to a nanoparticle, as nanosensors, in order to expand the ratiometric specific optical monitoring of various metabolic products: CO₂, NH₃, lactic acid etc. Intracellular fluorescence measurements are extremely useful in basic research of the physiological mechanisms of stimulation, e.g. for calcium mobilization and membrane depolarization. However, under the homeostatic cellular response, these intracellular stimulation signals become transient. Therefore, they are considered much less suitable for sensitive monitoring of PBLs stimulation, compared to the ongoing accumulative extracellular acidification that is recorded in the MA test. Such extracellular monitoring may be better facilitated by attachment of ratiometric molecular optical probes to nanoparticles. Extracellular monitoring is biocompatible, minimizing negative effects common to intracellular probes measurements, pointing on the advantage of the extracellular methods not only in basic research but also for various clinical applications in different cell types.

Any of the above acidification profiles can be used as an indicator of the metabolic activity of the cell. Alternatively, only one of the measured profiles is indicative of the metabolic activity of the cell or TIL.

As mentioned, the metabolic activity of the cell can be measured in naïve cells or activated/effector cells which have been exposed to different concentrations of a stimulant or an inhibitor. As used herein a “stimulant” or an “inhibitor” refers to an entity that increases, decreases or changes a metabolic pathway of a cell in response thereto.

In some embodiments, the stimulant is an antigen that is recognized by the TCR or BCR and leads to clonal expansion or antibody production. In some embodiments, specific stimulants or inhibitors are selected from PHA (in a range of 0.4-50 μg/ml), CONA (in a range of 0.8-100 μg/ml), PMA (in a range of 0.03-10 μg/ml), LPS (in a range of 0.03-10 μg/ml), MBP (in a range of 0.4-50 μg/ml), MelanA (in a range of 0.08-1 μg/ml), PSA (in a range of 0.4-50 μg/ml), Glucose (in a range of 0.4-5 μg/ml), L-glutamine (in a range of 0.03-4 μg/ml) and Rapamycin (in a range of 0.6-50 μg/ml).

In some embodiments, the stimulant or inhibitor may be a cell or cell-associated stimulant or inhibitor. Examples of stimulating cells include, but are not limited to, leukocytes, stem cells, platelets, red blood cells, bacteria, and fungi. Such a cellular stimulant or inhibitor may refer to an intact cell or a cell fragment e.g., cell membrane.

Alternatively, the stimulant or inhibitor may be cell-free such as a cell-free antigen (e.g., soluble antigen, virus, a cellular biological fluid). Specific examples of cell-free stimulants or inhibitors, include, but are not limited to, metabolites, nutrients (e.g., glucose), mitogens, peptides, cytokines, hormones, vitamins, drugs, antibodies, neurotransmitters, cancer specific antigens and various disease-associated tissue-specific normal antigens (TNAs).

Specific examples of MHC-restricted antigens (peptides) include but are not limited to CEA (Carcinoembryonic Antigen), MUC-1, HER2, CD340, MAGE and prolactin. In some embodiments, the stimulant or inhibitor is contacted with the cell at various concentrations.

It will be appreciated that one of more stimulants (e.g., 2, 3, 4) at the same or different concentrations can be used in the methods described herein. For example, different Her2 peptides or different stimulants all together e.g., PMA and Her2 peptides can be used.

In some embodiments, the MA test, as described above can be performed on a limited number of samples (e.g., using a tissue culture dish) or on a plurality of samples, screening response to a plurality of stimulants/inhibitors or screening a plurality of samples from different patients or a combination of same. High throughput screening can be performed using a multi well plate, a multi well plate reader (for detecting the fluorescent signal), a CCD camera applying image analysis or fiber optics matrices.

In some embodiments, in order to predict the efficacy of a TIL population, the acidification profiles of said TILs is compared to a control acidification profile of TILs obtained under identical conditions. Once the acidification profiles are obtained (e.g., with or without stimulant/inhibitor), the profile(s) are recorded. A difference in the metabolic activity between the TILs and the control (normal, unaffected), as evidenced from the acidification profiles obtained under identical conditions, is indicative of a disease associated with the modified metabolic activity profiles.

The results of the metabolic activity assay may be subject to decision tree models which classify the results and assist in determining the efficacy of the TILs. According to a preferred embodiment, at least two models are combined. Examples of such models include, but are not limited to, CHAID, C5 and C&R Tree. The Logistic model may be further applied.

In some embodiments, TILs are harvested when the MA is above a predetermined level. Any number of cell cultures may be examined in parallel for anti-tumor activity. In some embodiments, one cell culture is examined for anti-tumor activity. In some embodiments, two cell cultures are examined in parallel for anti-tumor activity. In some embodiments, three cell cultures are examined in parallel for anti-tumor activity. In some embodiments, four cell cultures are examined in parallel for anti-tumor activity. In some embodiments, five cell cultures are examined in parallel for anti-tumor activity. In some embodiments, more than five cell cultures are examined in parallel for anti-tumor activity.

In embodiments in which two or more cultures are examined, the selected cultures may be later combined and the number of TILs further expanded. In some embodiments in which two or more cultures are examined, each examined culture is separately expanded in separate cultures. In some embodiments, expanding multiple examined cultures separately advantageously increases lymphocyte diversity for patient treatment.

A skilled artisan would appreciate that treating a subject with a TIL population particularly efficient for lysing a tumor allows reducing the dose of TILs.

In some embodiments, TILs are produced from a biopsy. In some embodiments, TILs are produced from a resected tumor. In some embodiments, TILs are produced from an ablated tumor or from the tissue adjacent to it. A skilled artisan would appreciate that tumor ablation is a technique to burn, freeze, or kill a tumor, and that a number of technologies exist and are being developed to that purpose. The TILs population described herein can be produced from a tumor or a tissue ablated by any of these technologies. The TILs population described herein can be produced from a resected tumor.

In some embodiments, a population of TILs is produced by culturing and expanding TILs extracted from a resected tumor. In some embodiments, a population of TILs is produced by culturing and expanding TILs extracted from an ablated tumor or from the tissues adjacent to it. In some embodiments, said tumor is selected from a group comprising a carcinoma, a sarcoma, a lymphoma, leukemia, a germ cell tumor, a blastoma, chondrosarcoma, Ewing's sarcoma, malignant fibrous histiocytoma of bone/osteosarcoma, osteosarcoma, rhabdomyosarcoma, heart tumor, brain tumor, astrocytoma, glioma, medulloblastoma, neuroblastoma, breast tumor, medullary carcinoma, adrenocortical carcinoma, thyroid cancer, Merkel cell carcinoma, eye tumor, gastrointestinal tumor, colon tumor, gallbladder tumor, gastric (stomach) tumor, gastrointestinal carcinoid tumor, hepatocellular tumor, pancreatic tumor, rectal tumor, bladder tumor, cervical tumor, endometrial tumor, ovarian tumor, renal cell tumor, prostate tumor, testicular tumor, urethral tumor, uterine sarcoma, vaginal tumor, head tumor, neck tumor, nasopharyngeal carcinoma, hematopoietic tumor, lymphoma, Non-hodgkin lymphoma, skin tumor, basal-cell carcinoma, melanoma, small cell lung tumor, non-small cell lung tumor, or any combination thereof. In some embodiments, a population of TILs is produced by culturing and expanding TILs extracted from ablated melanoma. In some embodiments, a population of TILs is produced by culturing and expanding TILs extracted from resected melanoma.

### Stimulants

In some embodiments, the methods disclosed herein comprise contacting TILs with one or more stimulants. In some embodiments, the methods disclosed herein comprise in vitro contacting TILs with one or more stimulants. In some embodiments, the methods disclosed herein comprise contacting TILs with one or more stimulants and measuring metabolic activity of contacted TILs. In some embodiments, stimulated TILs have increased metabolic activity. In some embodiments, stimulated TILs have enhanced potency for cell therapy, as compared with TILs not contacted with one or more stimulants. In some embodiments, the metabolic activity of contacted TILs shows a shift in the metabolic activity towards that of a normal healthy cell sample. In some embodiments, the metabolic activity of contacted TILs shows a shift in the metabolic activity towards increased glycolysis cycle, as compared with TILs not contacted with one or more stimulants. In some embodiments, the metabolic activity of contacted TILs shows increased glycolysis cycle, as compared with TILs not contacted with one or more stimulants. In some embodiments, the metabolic activity of contacted TILs shows a reduced oxidative phosphorylation cycle, as compared with TILs not contacted with one or more stimulants.

In some embodiments, a stimulant comprises a peptide. In some embodiments, a stimulant comprises a synthetic peptide. In some embodiments, the peptide comprises a disease-specific peptide. In some embodiments, the peptide comprises a cancer-specific peptide. In some embodiments, the stimulant comprises a peptide as disclosed herein in detail. In some embodiments, the stimulant is selected from the group consisting of NY-ESO-1, Her-2a, ConA, PHA, MAGE-A3 and glucose.

In some embodiments, the stimulant comprises a partial peptide. In some embodiments, the stimulant comprises a short peptide. In some embodiments, the peptide comprises less than 30 amino acids. In some embodiments, the peptide comprises less than 25 amino acids. In some embodiments, the peptide comprises less than 20 amino acids. In some embodiments, the peptide comprises 20 amino acids.

In some embodiments, the stimulant is selected from the group consisting of CEA, MUC-1, NY-ESO-1, Her-2a, CON A, PHA, MAGE-A3, Cytokeratin 19, GRP, PMA, LPS, MelanA, PSA, MBP, Rapamycin, L-glutamine and Glucose.

In some embodiments, the stimulant is Phytohaemagglutinin (PHA). In some embodiments, the concentration of PHA is in a range of 0-100 μg/ml. In some embodiments, the stimulant is Concanavalin A (CON A). In some embodiments, the concentration of CON A is in a range of 0-100 μg/ml. In some embodiments, the stimulant is Phorbol Myristate Acetate (PMA). In some embodiments, the concentration of PMA is in a range of 0-10 ng/ml. In some embodiments, the stimulant is Lipopolysaccharide (LPS). In some embodiments, the concentration of LPS is in a range of 0-10 ng/ml. In some embodiments, the stimulant is Myelin-Basic-Protein (MBP). In some embodiments, the concentration of MBP is in a range of 0-100 μg/ml. In some embodiments, the stimulant is MelanA. In some embodiments, the concentration of MelanA is in a range of 0-1 μg/ml. In some embodiments, the stimulant is PSA. In some embodiments, the concentration of PSA is in a range of 0-50 μg/ml. In some embodiments, the stimulant is Glucose. In some embodiments, the concentration of Glucose is in a range of 0-10 mM. In some embodiments, the stimulant is L-glutamine. In some embodiments, the concentration of L-glutamine is in a range of 0-10 mM. In some embodiments, the stimulant is Rapamycin. In some embodiments, the concentration of Rapamycin is in a range of 0-50 mM. In some embodiments, the stimulant is Carcinoembryonic antigen (CEA). In some embodiments, the concentration of CEA is in a range of 0-100 μg/ml. In some embodiments, the stimulant is Mucin 1 (MUC-1). In some embodiments, the concentration of MUC-1 is in a range of 0-100 μg/ml. In some embodiments, the stimulant is New York esophageal squamous cell carcinoma 1 (NY-ESO-1). In some embodiments, the concentration of NY-ESO-1 is in a range of 0-100 μg/ml. In some embodiments, the stimulant is Cytokeratin 19. In some embodiments, the concentration of Cytokeratin 19 is in a range of 0-1 μg/ml. In some embodiments, the stimulant is Melanoma-associated antigen A3 (MAGE-A3). In some embodiments, the concentration of MAGE-A3 is in a range of 0-100 μg/ml. In some embodiments, the stimulant is Gastrin-releasing peptide (GRP). In some embodiments, the concentration of GRP is in a range of 0-100 μg/ml. In some embodiments, the stimulant is Her-2a. In some embodiments, the concentration of Her-2A is in a range of 0-100 μg/ml.

In some embodiments, the measuring is affected in an air-exposed chamber when the stimulant is NY-ESO-1. In some embodiments, the measuring is affected in an air-exposed chamber when the stimulant is Her-2a. In some embodiments, the measuring is affected in an air-exposed chamber when the stimulant is ConA. In some embodiments, the measuring is affected in an air-sealed chamber when the stimulant is PHA. In some embodiments, the measuring is affected in an air-sealed chamber when the stimulant is MAGE-A3. In some embodiments, the measuring is affected in an air-sealed chamber when the stimulant is glucose.

In some embodiments, the stimulant is selected from the group consisting of Pro-SFTPB, DAS, GAGE, HuD, AKAP4, PTTG1, Annexin I, 14-3-3 z, and 14-3-3 s. In some embodiments, the stimulant is Pro-surfactant protein B (Pro-SFTPB). In some embodiments, the stimulant is Diacetylspermine (DAS). In some embodiments, the stimulant is G antigen (GAGE, CTA4). In some embodiments, the stimulant is HuD. In some embodiments, the stimulant is A Kinase Anchor Protein 4 (AKAP4, CTA99). In some embodiments, the stimulant is Pituitary Tumor-Transforming 1 (PTTG1, Securin). In some embodiments, the stimulant is Annexin I. In some embodiments, the stimulant is 14-3-3 z or 14-3-3 s.

### Methods for Treating a Tumor

In some embodiments, disclosed herein is a method for treating a tumor in a subject in need thereof, the method comprising administering tumor infiltrating lymphocytes (TILs) produced by a method comprising:
a. extracting a biopsy comprising a tumor and/or its surroundings;
b. isolating, culturing, and expanding TILs isolated from said biopsy;
c. determining the metabolic activity (MA) in the TILs from step (b);
d. harvesting TILs from step (c); and
e. administering the subject with the TILs from step (d).

In some embodiments, TILs are harvested when the MA is above a predetermined threshold.

In some embodiments, the metabolic activity (MA) is determined by a method comprising measuring in the extracellular environment of the TILs the time-dependent acidification profiles due to secretion of:
(i) non-volatile soluble metabolic products and volatile soluble metabolic products;
(ii) non-volatile soluble metabolic products; and
(iii) volatile soluble metabolic products.

In some embodiments, measuring the acidification profile of (ii) is affected in an air-exposed chamber. In some embodiments, measuring the acidification profile of (i) is affected in an air-sealed chamber. In some embodiments, measuring the acidification profile of (iii) is affected by subtracting the acidification profile of (ii) from the acidification profile of (i). In some embodiments, time dependent acidification profiles are indicative of the metabolic activity of the cell.

In some embodiments, disclosed herein is a method for treating a tumor in a subject in need thereof, said treating comprising administering tumor infiltrating lymphocytes (TILs) produced by a method comprising:
a. extracting a biopsy comprising a tumor and/or its surroundings;
b. isolating, culturing, and expanding TILs isolated from said biopsy;
c. determining the metabolic activity (MA) in said TILs by a method comprising measuring in the extracellular environment of the cell the time-dependent acidification profiles due to secretion of:
   (i) non-volatile soluble metabolic products and volatile soluble metabolic products;
   (ii) non-volatile soluble metabolic products; and
   (iii) volatile soluble metabolic products;
wherein said measuring acidification profile of (ii) is affected in an air-exposed chamber, and
wherein said measuring acidification profile of (i) is affected in an air-sealed chamber, and
wherein said measuring acidification profile of (iii) is affected by subtracting the acidification profile of (ii) from the acidification profile of (i), and
wherein all of said time dependent acidification profiles are indicative of the metabolic activity of the cell;
d. harvesting TILs from step (c); and
e. administering the subject with the TILs from step (d).

In some embodiments, TILs are harvested when the level of the phenotype parameter is above, or below, a pre-determined threshold.

In some embodiments, treating a tumor comprises decreasing the size of the tumor. In some embodiments, treating a tumor comprises eliciting an enhanced immune response against the tumor. In some embodiments, treating a tumor comprises delaying metastasis. In some embodiments, treating a tumor comprises increasing survival of a patient. In some embodiments, treating a tumor comprises increasing the relapse time, or disease free survival (DFS) time. In some embodiments, treating a tumor comprises increasing progression free survival (PFS) time. In some embodiments, treating a tumor comprises increasing the quality of life of a patient.

The methods and the TILs disclosed herein are useful for the treatment of a variety of cancers or tumors. In some embodiments, the cancer or tumor comprises a solid tumor. In some embodiments, the cancer or tumor comprises a non-solid tumor. In some embodiments, the cancer or tumor comprises a metastasis of a cancer or tumor.

In some embodiments the method for the treatment of a cancer is directed to cancers including, but not limited to, melanoma, non-Hodgkin's lymphoma, Hodgkin's disease, leukemia, plasmocytoma, sarcoma, glioma, thymoma, breast cancer, prostate cancer, colo-rectal cancer, kidney cancer, renal cell carcinoma, uterine cancer, pancreatic cancer, esophageal cancer, brain cancer, lung cancer, ovarian cancer, cervical cancer, testicular cancer, gastric cancer, esophageal cancer, multiple myeloma, hepatoma, acute lymphoblastic leukemia (ALL), acute myelogenous leukemia (AML), chronic myelogenous leukemia (CML), and chronic lymphocytic leukemia (CLL).

Solid tumors may be benign (not cancer), or malignant (cancer). Different types of solid tumors are named for the type of cells that form them. Examples of solid tumors for which treatment may be provided include sarcomas, carcinomas, and lymphomas. In some embodiments, solid tumors for which treatment may be provided include neoplasms (new growth of cells) or lesions (damage of anatomic structures or disturbance of physiological functions) formed by an abnormal growth of body tissue cells other than blood, bone marrow or lymphatic cells. In some embodiments, a solid tumor for which treatment may be provided consists of an abnormal mass of cells which may stem from different tissue types such as liver, colon, breast, or lung, and which initially grows in the organ of its cellular origin. However, such cancers may spread to other organs through metastatic tumor growth in advanced stages of the disease.

In some embodiments of a method for treatment of a cancer or tumor, the solid tumor or cancer comprises a sarcoma or a carcinoma, adrenocortical tumor (adenoma and carcinoma), a fibrosarcoma, a myxo-sarcoma, a liposarcoma, a chondrosarcoma, an osteogenic sarcoma, a chordoma, an angiosarcoma, an endothelio sarcoma, a lymphangiosarcoma, a lymphangioendothelio sarcoma, a synovioma, a mesothelioma, an Ewing's tumor, a leiomyosarcoma, a rhabdomyosarcoma, a colon carcinoma, a pancreatic cancer or tumor, a breast cancer or tumor, an ovarian cancer or tumor, a prostate cancer or tumor, a squamous cell carcinoma, a squamous cell carcinoma of the lung, a basal cell carcinoma, an adenocarcinoma, a sweat gland carcinoma, a sebaceous gland carcinoma, a papillary carcinoma, a papillary adenocarcinomas, a cystadenocarcinoma, a medullary carcinoma, a bronchogenic carcinoma, a renal cell carcinoma, a hepatoma, a bile duct carcinoma, a choriocarcinoma, a seminoma, an embryonal carcinoma, a colorectal carcinoma, a desmoid tumor, a desmoplastic small round cell tumor, an endocrine tumor, a germ cell tumor, a hepatoblastoma, a hepatocellular carcinoma, a melanoma, a neuroblastoma, an osteosarcoma, a retinoblastoma, a rhabdomyosarcoma, a soft tissue sarcoma other than rhabdomyosarcoma, a Wilms, Tumor, a cervical cancer or tumor, a uterine cancer or tumor, a testicular cancer or tumor, a lung carcinoma, a small cell lung carcinoma, an anal cancer, a glioblastoma, an epithelial tumor of the head and neck, a bladder carcinoma, an epithelial carcinoma, a glioma, an astrocytoma, a medulloblastoma, a craniopharyngioma, an ependymoma, a pinealoma, a hemangioblastoma, an acoustic neuroma, an oligodenroglioma, a schwannoma, a meningioma, a melanoma, a neuroblastoma, or a retinoblastoma.

In some embodiments of a method for treatment of a cancer or tumor, the tumor or cancer comprises a non-solid tumor, that is a non-solid cancer. In some embodiments methods for treatment of a cancer or tumor may be for a diffuse cancer, wherein the cancer is widely spread; not localized or confined. In some embodiments, a diffuse cancer may comprise a non-solid tumor. Examples of diffuse cancers include leukemias. Leukemias comprise a cancer that starts in blood-forming tissue, such as the bone marrow, and causes large numbers of abnormal blood cells to be produced and enter the bloodstream.

In some embodiments of a method for treatment of a cancer or tumor, the diffuse cancer comprises a B-cell malignancy. In some embodiments, the diffuse cancer comprises leukemia. In some embodiments, the cancer is lymphoma. In some embodiments, the lymphoma is large B-cell lymphoma.

In some embodiments of a method for treatment of a cancer or tumor, the diffuse cancer or tumor comprises a hematological tumor. In some embodiments, hematological tumors are cancer types affecting blood, bone marrow, and lymph nodes. Hematological tumors may derive from either of the two major blood cell lineages: myeloid and lymphoid cell lines. The myeloid cell line normally produces granulocytes, erythrocytes, thrombocytes, macrophages, and masT-cells, whereas the lymphoid cell line produces B, T, NK and plasma cells. Lymphomas (e.g., Hodgkin's Lymphoma), lymphocytic leukemias, and myeloma are derived from the lymphoid line, while acute and chronic myelogenous leukemia (AML, CML), myelodysplastic syndromes and myeloproliferative diseases are myeloid in origin.

In some embodiments of a method for treatment of a cancer or tumor, the non-solid (diffuse) cancer or tumor comprises a hematopoietic malignancy, a blood cell cancer, a leukemia, a myelodysplastic syndrome, a lymphoma, a multiple myeloma (a plasma cell myeloma), an acute lymphoblastic leukemia, an acute myelogenous leukemia, a chronic myelogenous leukemia, a Hodgkin lymphoma, a non-Hodgkin lymphoma, or plasma cell leukemia.

In some embodiments, the method for treating a tumor further comprises any other cancer treatment. In some embodiments, the method for treating a tumor further comprises surgery. In some embodiments, the method for treating a tumor further comprises radiation therapy. In some embodiments, the method for treating a tumor further comprises chemotherapy. In some embodiments, the method for treating a tumor further comprises immunotherapy. In some embodiments, the method for treating a tumor further comprises a cell therapy.

In some embodiments, the method for treating a tumor further comprises targeted therapy. In some embodiments, the method for treating a tumor further comprises hormone therapy. In some embodiments, the method for treating a tumor further comprises stem cell transplant. In some embodiments, the method for treating a tumor further comprises personalized medicine or precision medicine treatment.

In some embodiments, the method for treating a tumor disclosed herein further comprises contacting TILs with one or more stimulants. In some embodiments, the method for treating a tumor disclosed herein further comprises contacting TILs with one or more stimulants, and selecting and expanding TILs having enhanced potency for cell therapy based on the metabolic activity. In some embodiments, the method for treating a tumor disclosed herein further comprises selecting and expanding TILs having enhanced potency for cell therapy based on the metabolic activity. In some embodiments, the method for treating a tumor disclosed herein further comprises selecting and expanding TILs having increased metabolic activity. In some embodiments, selecting and expanding TILs having increased metabolic activity occurs prior to harvesting TILs. In some embodiments, TILs having enhanced potency for cell therapy have increased metabolic activity (MA). In some embodiments, contacting TILs with one or more stimulants occurs prior to, or concomitant with determining the metabolic activity. In some embodiments, contacting TILs with one or more stimulants occurs prior to determining the metabolic activity. In some embodiments, contacting TILs with one or more stimulants occurs concomitant with determining the metabolic activity.

In some embodiments, the methods of treatment disclosed herein further comprise contacting TILs with one or more stimulants. In some embodiments, the methods of treatment disclosed herein further comprise measuring metabolic activity of contacted TILs. In some embodiments, stimulated TILs have increased metabolic activity. In some embodiments, stimulated TILs have enhanced potency for cell therapy, as compared with TILs not contacted with one or more stimulants. In some embodiments, the metabolic activity of contacted TILs shows a shift in the metabolic activity towards that of a normal healthy cell sample. In some embodiments, the metabolic activity of contacted TILs shows a shift in the metabolic activity towards increased glycolysis cycle, as compared with TILs not contacted with one or more stimulants. In some embodiments, the metabolic activity of contacted TILs shows increased glycolysis cycle, as compared with TILs not contacted with one or more stimulants. In some embodiments, the metabolic activity of contacted TILs shows a reduced oxidative phosphorylation cycle, as compared with TILs not contacted with one or more stimulants.

In some embodiments, a stimulant comprises a peptide. In some embodiments, a stimulant comprises a synthetic peptide. In some embodiments, the peptide comprises a disease-specific peptide. In some embodiments, the peptide comprises a cancer-specific peptide. In some embodiments, the stimulant comprises a peptide as disclosed herein in detail. In some embodiments, the stimulant is selected from the group consisting of NY-ESO-1, Her-2a, ConA, PHA, MAGE-A3 and glucose.

In some embodiments, the method for treating a tumor further comprises administering to the subject a growth factor that promotes the growth and activation of TILs. In some embodiments, the method for treating a tumor further comprises administering to the subject a T-cell growth factor that promotes the growth and activation of TILs. In some embodiments, the growth factor is administered to the subject concomitantly with the TILs or subsequently to the TILs. In some embodiments, the growth factor is administered to the subject subsequently to the TILs. In some embodiments, the growth factor is administered to the subject concomitantly with the TILs. The growth factor can be any suitable growth factor that promotes the growth and activation of the TILs.

In some embodiments, the method further comprises administering IL-2 to the subject. In some embodiments, the method further comprises administering IL-7 to the subject. In some embodiments, the method further comprises administering IL-15 to the subject. In some embodiments, the method further comprises administering IL-12 to the subject. In some embodiments, the method further comprises administering various growth factor combinations, for example IL-2 and IL-7, IL-2 and IL-15, IL-7 and IL-15, IL-2, IL-7 and IL-15, IL-12 and IL-7, IL-12 and IL-15, or IL-12 and IL-2.

In some embodiments, any dose of TILs can be administered to the subject. In some embodiments, a dose between about 0.1×10¹⁰ TILs and 100×10¹⁰ TILs are administered. In some embodiments, a dose between about 0.5×10¹⁰ TILs and 50×10¹⁰ TILs are administered. In some embodiments, a dose between about 1×10¹⁰ TILs and 10×10¹⁰ TILs are administered. In some embodiments, a dose of about 0.5×10¹⁰ TILs are administered. In some embodiments, a dose of about 1×10¹⁰ are administered. In some embodiments, a dose of about 2.5×10¹⁰) TILs are administered. In some embodiments, a dose of about 5×10¹⁰ TILs are administered. In some embodiments, a dose of about 10×10¹⁰ TILs are administered. In some embodiments, a dose of about 25×10¹⁰ TILs are administered. In some embodiments, a dose of about 50×10¹⁰ TILs are administered.

It will be appreciated that TILs can be provided to the subject with additional active agents to achieve an improved therapeutic effect as compared to treatment with TILs alone. In some embodiments, additional active agents include anti-cancer drugs.

In some embodiments, an anti-cancer drug comprises cytotoxic antineoplastics. In some embodiments, an anti-cancer drug comprises nucleoside analogues. In some embodiments, a nucleoside analogue is selected from the group comprising azacitidine, capecitabine, carmofur, cladribine, clofarabine, cytarabine, decitabine, floxuridine, fludarabine, fluorouracil, gemcitabine, mercaptopurine, nelarabine, pentostatin, tegafur, or tioguanine.

In some embodiments, an anti-cancer drug comprises antifolates, methotrexate, pemetrexed, raltitrexed, antimetabolites, hydroxycarbamide, topoisomerase inhibitors, irinotecan, topotecan, anthracyclines, daunorubicin, doxorubicin, epirubicin, idarubicin, mitoxantrone, valrubicin, podophyllotoxins, etoposide, teniposide, taxanes, cabazitaxel, docetaxel, paclitaxel, vinca alkaloids, vinblastine, vincristine, vindesine, vinflunine, vinorelbine, alkylating agents, bendamustine, busulfan, carmustine, chlorambucil, chlormethine, cyclophosphamide, dacarbazine, fotemustine, ifosfamide, lomustine, melphalan, streptozotocin, temozolomide, platinum compounds, carboplatin, cisplatin, nedaplatin, or oxaliplatin.

In some embodiments, an anti-cancer drug comprises altretamine, bleomycin, bortezomib, dactinomycin, estramustine, ixabepilone, mitomycin, procarbazine, targeted antineoplastics, monoclonal antibodies, alemtuzumab, bevacizumab, cetuximab, denosumab, gemtuzumab ozogamicin, ibritumomab tiuxetan, ipilimumab, nivolumab, ofatumumab, panitumumab, pembrolizumab, pertuzumab, rituximab, tositumomab, trastuzumab, tyrosine kinase inhibitor, afatinib, aflibercept, axitinib, bosutinib, crizotinib, dasatinib, erlotinib, gefitinib, imatinib, lapatinib, nilotinib, pazopanib, ponatinib, regorafenib, ruxolitinib, sorafenib, sunitinib, vandetanib, mTOR inhibitors, everolimus, temsirolimus, retinoids, alitretinoin, bexarotene, isotretinoin, tamibarotene, tretinoin, immunomodulatory agents (iMiDs), lenalidomide, pomalidomide, thalidomide, histone deacetylase inhibitors, panobinostat, romidepsin, valproate, vorinostat, anagrelide, arsenic trioxide, asparaginase, BCG vaccine, denileukin diftitox, and vemurafenib.

In some embodiments, disclosed herein is a method of monitoring treatment, the method comprising:
(a) treating a subject in need with TILs; and
(b) measuring metabolic activity in TILs of the subject, wherein a shift in the metabolic activity of the TILs towards that of a normal healthy cell sample examined under identical conditions is indicative of an efficacious treatment of the disease. In some embodiments, the treatment is cancer treatment.

In some embodiments, disclosed herein is a method of measuring the efficacy of therapy, the method comprising:
(a) treating a subject in need with TILs; and
(b) measuring metabolic activity in TILs of the subject, wherein a shift in the metabolic activity of the TILs towards that of a normal healthy cell sample examined under identical conditions is indicative of an efficacious therapy. In some embodiments, the therapy is cancer therapy.

In some embodiments, disclosed herein is a method of monitoring treatment of a subject, the method comprising:
(a) treating a subject in need with TILs; and
(b) measuring metabolic activity in TILs of the subject, wherein a shift in the metabolic activity of the TILs towards an increased glycolysis cycle is indicative of an efficacious treatment of the disease.

In some embodiments, disclosed herein is a method of measuring the efficacy of therapy, the method comprising:
(a) treating a subject in need with TILs; and
(b) measuring metabolic activity in TILs of the subject, wherein a shift in the metabolic activity of the TILs towards an increased glycolysis cycle is indicative of an efficacious therapy.

Any of the methods described herein can be used in a diagnostic kit aimed for executing the method.

In some embodiments, described herein are compositions comprising the TILs population as described herein and administration of such compositions in a variety of therapeutic settings.

### Pharmaceutical Compositions

Administration of the TILs population described herein, in pure form or in an appropriate pharmaceutical composition, can be carried out via any of the accepted modes of administration of agents for serving similar utilities. The pharmaceutical compositions comprising TILs or other pharmaceutically active ingredients (including other anti-cancer agents as described elsewhere herein) can be prepared by combining TILs population or other anti-cancer agent with an appropriate physiologically acceptable carrier, diluent or excipient, and may be formulated into preparations in solid, semi-solid, or liquid forms, such as tablets, capsules, powders, granules, ointments, solutions, suppositories, injections, inhalants, gels, and microspheres. In addition, suitable excipients such as salts, buffers and stabilizers may be present within the composition.

Administration may be achieved by a variety of different routes, including oral, parenteral, nasal, intravenous, intradermal, subcutaneous or topical. In some embodiments, modes of administration depend upon the nature of the condition to be treated or prevented. An amount that, following administration, reduces, inhibits, prevents or delays the progression and/or metastasis of a cancer is considered effective. A skilled artisan would appreciate that the term “physiologically acceptable carrier, diluent or excipient”, may in some embodiments be used interchangeably with the term “pharmaceutically acceptable carrier” having all the same means and qualities.

In some embodiments, a method of treating, preventing, inhibiting the growth of, delaying disease progression, reducing the tumor load, or reducing the incidence of a cancer or a tumor in a subject, or any combination thereof, comprising a step of resecting a tumor, and a step of administering a pharmaceutical composition comprising TILs population produced according to the methods described herein, wherein the method treats, prevents, inhibits the growth of, delays the disease progression, reduces the tumor load, or reduces the incidence of the cancer or a tumor in said subject, or reduces the minimal residual disease, increases remission, increases remission duration, reduces tumor relapse rate, prevents metastasis of said tumor or said cancer, or reduces the rate of metastasis of said tumor or said cancer, or any combination thereof, compared with a subject not administered said pharmaceutical composition.

A skilled artisan would appreciate that the term “treating” and grammatical forms thereof, may in some embodiments encompass both therapeutic treatment and prophylactic or preventative measures with respect to a tumor or cancer as described herein, wherein the object is to prevent or lessen the targeted tumor or cancer as described herein. Thus, in some embodiments of methods disclosed herein, treating may include directly affecting or curing, suppressing, inhibiting, preventing, reducing the severity of, delaying the onset of, reducing symptoms associated with the cancer or tumor. Thus, in some embodiments, “treating” encompasses preventing, delaying progression, inhibiting the growth of, delaying disease progression, reducing tumor load, reducing the incidence of, expediting remission, inducing remission, augmenting remission, speeding recovery, increasing efficacy of or decreasing resistance to alternative therapeutics, or a combination thereof. In some embodiments, “preventing” encompasses delaying the onset of symptoms, preventing relapse to a disease, decreasing the number or frequency of relapse episodes, increasing latency between symptomatic episodes, or a combination thereof. In some embodiments, “suppressing” or “inhibiting”, encompass reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

In some embodiments, the subject in need is a human subject. In some embodiments, the subject in need is a human child. In some embodiments, the subject in need is an adult human. In some embodiments, the subject in need is a human infant. In some embodiments, the subject is a non-human mammal.

In some embodiments, the number of TILs administered is sufficient to result in tumor regression, as indicated by a statistically significant decrease in the amount of viable tumor, for example, at least a 50% decrease in tumor mass, or by altered (e.g., decreased with statistical significance) scan dimensions. In other embodiments, the number of TILs administered is sufficient to result in clinically relevant reduction in disease symptoms as would be known to the skilled clinician.

The precise dosage and duration of treatment is a function of the cancer and/or tumor conditions and may be determined empirically using known testing protocols or by testing the compositions in model systems known in the art and extrapolating therefrom. Controlled clinical trials may also be performed. Dosages may also vary with the severity of the condition to be alleviated. A pharmaceutical composition is generally formulated and administered to exert a therapeutically useful effect while minimizing undesirable side effects. The composition may be administered one time or may be divided into a number of smaller doses to be administered at intervals of time. For any particular subject, specific dosage regimens may be adjusted over time according to the individual need.

The TILs-containing compositions may be administered alone or in combination with other known cancer treatments, such as radiation therapy, chemotherapy, transplantation, immunotherapy, hormone therapy, photodynamic therapy, etc.

Typical routes of administering TILs and/or other anti-cancer agents administered with them include, without limitation, oral, topical, transdermal, inhalation, parenteral, sublingual, buccal, rectal, vaginal, and intranasal. The term parenteral as used herein includes subcutaneous injections, intravenous, intramuscular, intrasternal injection or infusion techniques. Pharmaceutical compositions according to certain embodiments as described herein, are formulated so as to allow the active ingredients contained therein to be bioavailable upon administration of the composition to a patient. Compositions that will be administered to a subject or patient may take the form of one or more dosage units, where for example, a tablet may be a single dosage unit, and a container of a herein described anti-cancer agent may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain a therapeutically effective number of TILs of the present disclosure, for treatment of a disease or condition of interest in accordance with teachings herein.

A pharmaceutical composition may be in the form of a solid or liquid. In one embodiment, the pharmaceutically acceptable carrier(s) are particulate, so that the compositions are, for example, in tablet or powder form. The pharmaceutically acceptable carrier(s) may be liquid, with the compositions being, for example, an oral oil, injectable liquid or an aerosol, which is useful in, for example, inhalatory administration. When intended for oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer, or the like. Such a solid composition will typically contain one or more inert diluents or edible pharmaceutically acceptable carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent. When the pharmaceutical composition is in the form of a capsule, for example, a gelatin capsule, it may contain, in addition to materials of the above type, a liquid pharmaceutically acceptable carrier such as polyethylene glycol or oil.

The pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition intended for either parenteral or oral administration should contain an amount of the anti-cancer agents herein disclosed such that a suitable dosage will be obtained. When intended for oral administration, this amount may be varied to be between 0.1 and about 70% of the weight of the composition. Certain oral pharmaceutical compositions contain between about 4% and about 75% of the anti-cancer agents. In certain embodiments, pharmaceutical compositions and preparations according to the embodiments described herein, are prepared so that a parenteral dosage unit contains between 0.01 to 10% by weight of the anti-cancer agents.

The pharmaceutical composition may be intended for topical administration, in which case the pharmaceutically acceptable carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. The pharmaceutical composition may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter and polyethylene glycol.

The pharmaceutical composition may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule. The pharmaceutical composition in solid or liquid form may include an agent that binds to the antibody as disclosed herein, and thereby assists in the delivery of the compound. Suitable agents that may act in this capacity include other monoclonal or polyclonal antibodies, one or more proteins or a liposome. The pharmaceutical composition may consist essentially of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols may be delivered in single phase, bi-phasic, or tri-phasic systems in order to deliver the active ingredient(s). Delivery of the aerosol includes the necessary container, activators, valves, subcontainers, and the like, which together may form a kit. One of ordinary skill in the art, without undue experimentation may determine preferred aerosols.

The pharmaceutical compositions may be prepared by methodology well known in the pharmaceutical art. For example, a pharmaceutical composition intended to be administered by injection can be prepared by combining a composition that comprises TILs population and/or other anti-cancer agents as described herein and optionally, one or more of salts, buffers and/or stabilizers, with sterile, distilled water so as to form a solution. A surfactant may be added to facilitate the formation of a homogeneous solution or suspension. Surfactants are compounds that non-covalently interact with the active ingredients so as to facilitate dissolution or homogeneous suspension of the active ingredients in the aqueous delivery system.

In some embodiments, the pharmaceutical composition comprises 80% Plasma-lyte A+20% of human serum albumin (HSA) solution. In one embodiment, the human serum albumin (HSA) solution is 20% w/v.

The compositions may be administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy.

Compositions comprising TILs and/or anti-cancer agents described herein may also be administered simultaneously with, prior to, or after administration of one or more other therapeutic agents. Such combination therapy may include administration of a single pharmaceutical dosage formulation which contains a compound as disclosed herein, and one or more additional active agents, as well as administration of compositions comprising TILs as disclosed herein, and each active agent in its own separate pharmaceutical dosage formulation. For example, TILs population as described herein, and the other active agent can be administered to the patient together in a single parenteral dosage composition such as in a saline solution or other physiologically acceptable solution, or each agent administered in separate parenteral dosage formulations. Where separate dosage formulations are used, the compositions comprising TILs, and one or more additional active agents can be administered at essentially the same time, i.e., concurrently, or at separately staggered times, i.e., sequentially and in any order; combination therapy is understood to include all these regimens.

A variety of other therapeutic agents may be used in conjunction with the TILs population described herein. In one embodiment, the TILs population is administered with an anti-inflammatory agent. Anti-inflammatory agents or drugs include, but are not limited to, steroids and glucocorticoids (including betamethasone, budesonide, dexamethasone, hydrocortisone acetate, hydrocortisone, hydrocortisone, methylprednisolone, prednisolone, prednisone, triamcinolone), nonsteroidal anti-inflammatory drugs (NSAIDS) including aspirin, ibuprofen, naproxen, methotrexate, sulfasalazine, leflunomide, anti-TNF medications, cyclophosphamide and mycophenolate.

The compositions comprising the herein described TILs population may be administered to an individual afflicted with cancer and/or a tumor. For in vivo use for the treatment of human disease, the TILs population described herein is generally incorporated into a pharmaceutical composition prior to administration. A pharmaceutical composition comprises one or more of the TILs population described herein in combination with a pharmaceutically acceptable carrier or excipient as described elsewhere herein. To prepare a pharmaceutical composition, an effective amount of one or more of the TILs population is mixed with any pharmaceutically acceptable carrier(s) or excipient known to those skilled in the art to be suitable for the particular mode of administration.

A pharmaceutically acceptable carrier may be liquid, semi-liquid or solid. Solutions or suspensions used for parenteral, intradermal, subcutaneous or topical application may include, for example, a sterile diluent (such as water), saline solution, fixed oil, polyethylene glycol, glycerin, propylene glycol or other synthetic solvent; antimicrobial agents (such as benzyl alcohol and methyl parabens, phenols or cresols, mercurials, chlorobutanol, methyl and propyl p-hydroxybenzoic acid esters, thimerosal, benzalkonium chloride and benzethonium chloride); antioxidants (such as ascorbic acid and sodium bisulfite; methionine, sodium thiosulfate, platinum, catalase, citric acid, cysteine, thioglycerol, thioglycolic acid, thiosorbitol, butylated hydroxyanisol, butylated hydroxytoluene, and/or propyl gallate) and chelating agents (such as ethylenediaminetetraacetic acid (EDTA)); buffers (such as acetates, citrates and phosphates). If administered intravenously, suitable pharmaceutically acceptable carriers include physiological saline or phosphate buffered saline (PBS), and solutions containing thickening and solubilizing agents, such as glucose, polyethylene glycol, polypropylene glycol and mixtures thereof.

The compositions comprising TILs described herein may be prepared with pharmaceutically acceptable carriers that protect the TILs against rapid elimination from the body. Such pharmaceutically acceptable carriers include controlled release formulations, such as, but not limited to, implants and microencapsulated delivery systems, and biodegradable, biocompatible polymers, such as ethylene vinyl acetate, polyanhydrides, polyglycolic acid, polyorthoesters, polylactic acid and others known to those of ordinary skill in the art.

For any preparation used in the methods disclosed herein, the therapeutically effective amount or dose can be estimated initially from in vitro assays. For example, a dose can be formulated in animal models and such information can be used to determine useful doses more accurately in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. [See e.g., Fingl, et al., (1975) “The Pharmacological Basis of Therapeutics”, Ch. 1 p. 1].

Depending on the severity and responsiveness of the condition to be treated, dosing can be of a single or a plurality of administrations, with course of treatment lasting from several days to several weeks or until cure is affected or diminution of the disease state is achieved. The amount of a composition to be administered will, of course, be dependent on e.g. the subject being treated, the severity of the affliction, the manner of administration, the judgment of the prescribing physician, etc.

Compositions of some embodiments, may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation formulated in a compatible pharmaceutical carrier may also be prepared, placed in an appropriate container, and labeled for treatment of an indicated condition, as is further detailed above.

### EXAMPLES

### Example 1—Production of Tumor Infiltrating Lymphocyte (TILs)

### Generation of Pre-Rapid Expansion Protocol, Rapid Expansion (REP) Tumor Infiltrating Lymphocyte (TILs) Culture and Cell Harvest.

The isolation and expansion of tumor infiltrating lymphocyte (TILs) will be performed following protocols known in the art. The objective at the first stage of the protocol is generating a TIL culture.

Briefly, tumor tissue will be surgically removed from patients. Various techniques, such as homogenization, fragmentation, fine needle aspiration, and enzymatic digestion, will be used to obtain cancer cultures. TILs cultures will be established by dissecting tumor specimens into 1-2 mm³ pieces.

On pre-REP Days 9±1 and 12±1, the possibility of early harvest is evaluated according to lactate concentration, cell number, viability, and identity.

### Example 2—Characterization of TILs Subpopulation Composition Using Flow Cytometry

Various combinations of antibody staining will be used for subpopulation mapping. For flow cytometric analysis of cell surface receptors, 1.5×10⁵ cells will be washed and re-suspended in PBS containing 0.1% BSA. Cells will be incubated on ice with the appropriate conjugated antibodies for 20 min and will be subsequently washed three times with cold PBS containing 0.1% BSA. Background staining will be assessed using isotype control antibodies.

### Example 3—TILs Cytotoxicity Activity

TILs cytotoxic activity will be determined by a cytotoxicity assay, similarly to the well-established enzyme-linked ELISA immunoassay. TILs (effector cells) will be co-cultured with autologous cancer cells (target cells) or with beads coated with antibodies against CD3, CD28 and CD137 served as non-specific stimulator and mimic the stimulation of autologous cancer cells.

To determine the level of immune response and cytokine release, TILs will be mixed at effector-target in different ratios, in 96-well plate at 37° C., 5% CO2, and incubated for 5 h, in a PRIME-XV medium. Cultures will be terminated by centrifugation at 700 g for 10 minutes at 4° C. Levels of cytokines, such as interferon-γ (IFN-γ), Genzyme and lactate dehydrogenase (LDH), will be determined.

### Example 4—Growth of T Cells in High Density Culturing Conditions

### Objective: Growing T Cells in High Density Culturing Conditions.

Methods: T cell lymphocytes were grown on a 50 square cm bioreactor system with 150 ml media for 7 days, starting at a cell concentration of about 4 million cells per ml, which is the maximum known cell density for these cells. Based on knowledge in the art, this is the density at which T cells would normally be passaged and then maintained at 1 million cells per ml. Culture media was perfused. However, the volume of media in the chamber remained constant at 150 ml.

### Results

The results indicate that using the bioreactor and the protocols disclosed herein, T cells can be grown at a density (cells/ml) that is more than 24-fold of the normally expected density for T cells (1×10⁶/ml). Similarly, the data shows that growing cells in a bioreactor system having a footprint of 50 cm², that starting at 13.3 million per cm² (as opposed to the maximum reported of 10×10⁶/cm²), use of a bioreactor described herein resulted in having 73.6×10⁶ per cm².

Table 1 shows the number of T cells as a function of time in T when grown in a bioreactor.

TABLE 1

| Days | CM² | Cells(E6/ml) | Total Cells | Cells/cm2 |
|---|---|---|---|---|
| 0 | 50 | 3.580 | 667,000,000 | 13,340,000 |
| 2 | 50 | 5.230 | 784,500,000 | 15,690,000 |
| 4 | 50 | 9.267 | 1,390,050,000 | 27,801,000 |
| 7 | 50 | 24.55 | 3,683,632,500 | 73,672,650 |

Conclusion: T cells can be grown at high density using a bioreactor comprising a very small footprint (50 cm²) of the culturing system. Thus, the bioreactor provides for a system that allowed optimal and adaptive T cell culturing at changing volumes and feeding schemes, allowed for activating, manipulating, feeding, washing, and formulating cells in a closed automated manner with minimal sheer force. Additional cell incubators or centrifuges were not required for culturing and collection of cells, respectively.

### Example 5—Comparison of T Cell Cultures: Bioreactors Vs. Tissue Culture Flasks

Objective: Compare culturing T cells in a bioreactor comprising a 50 cm² perforated barrier with culturing cells in tissue culture flasks.

Methods: T cells were cultured for 14 days on a same dishes as follows: in either a 50 cm² bioreactor system with perfusion, or a T75 flask without media change, or T75 flask with media exchange every 4 days.

### Results

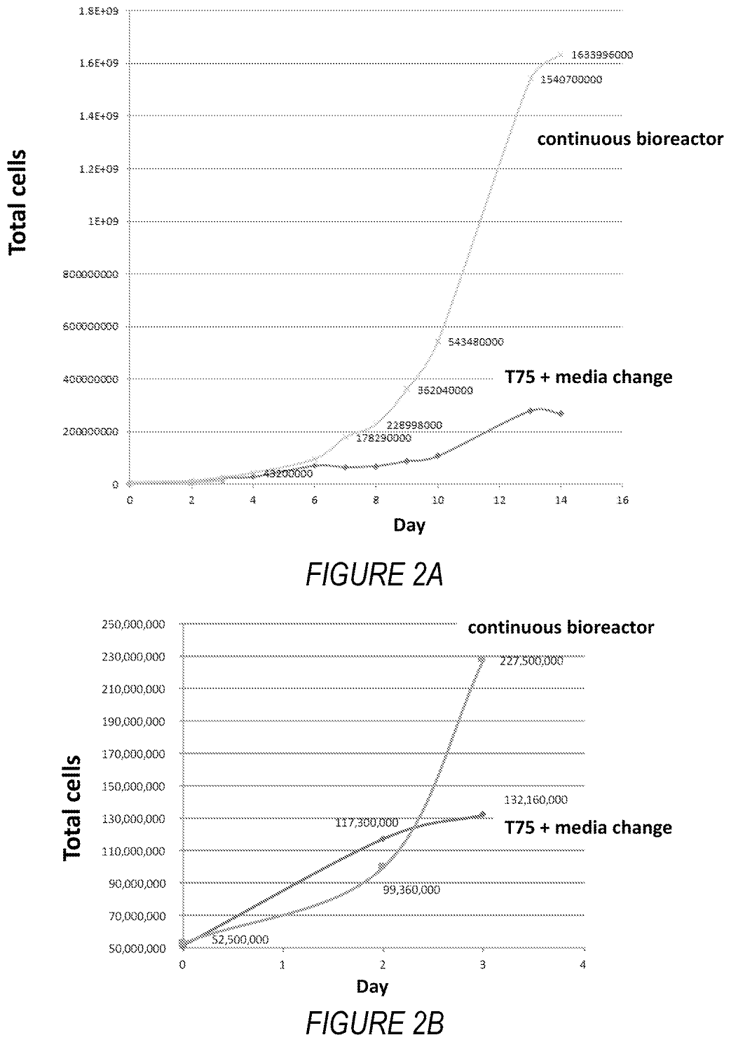

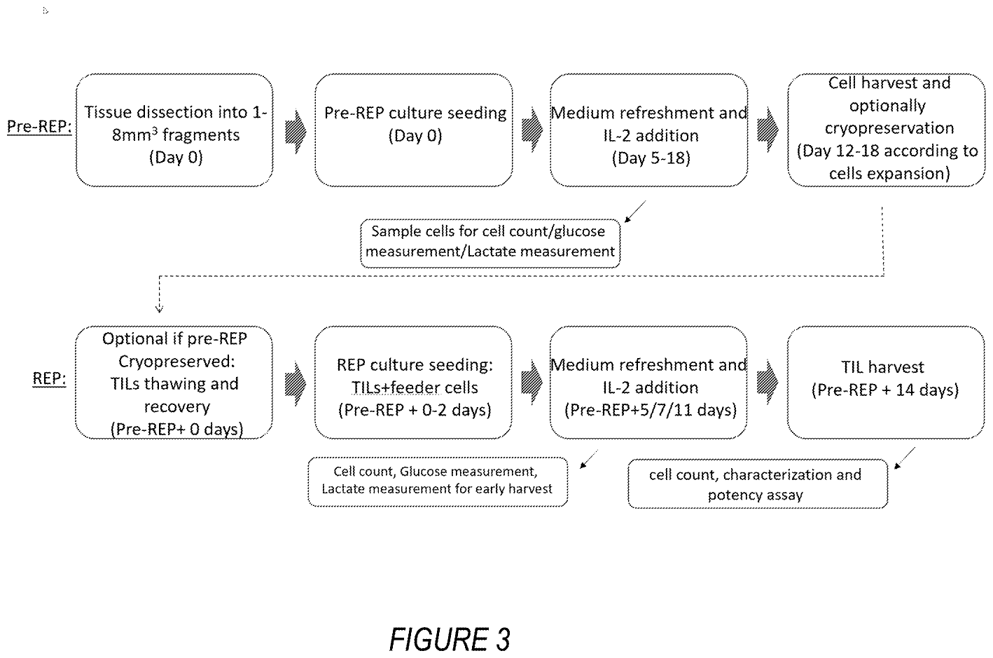

Cells grown in the closed continuous bioreactor system proliferated for 14 days, and achieved a total cell number of about 1634×10⁶ cells, compared with only about 4×10⁶ cells in the T75 flask without media change (data not shown), and only about 300×10⁶ cells in the T75 flask with media change (FIGS. 2A-2B). The 14-day time frame was used based on the fact that the number of cells in the bioreactor surpassed that in the flasks after a week. However, cells can be cultured for more than two weeks in the bioreactor (data not shown).

### CONCLUSION

Culture of T cells via the above mentioned methods described herein is more effective than culturing T cells in flasks even with media exchange.

### Example 6—Construction of an Algorithm for Identifying Tumor Infiltrating Lymphocytes (TILs) with High Metabolic Activity (MA)

Identification of tumor infiltrating lymphocytes (TILs) with high metabolic activity (MA) is performed according to the methods described in U.S. Pat. No. 8,728,758, which is incorporated herein by reference and is summarized below.

Each well in a black non-binding, low-volume 384 multi-well plate is loaded with 10 μl of a TILs solution and 10 μl of working solution plus HPTS including one of ten reagents in 8 increasing concentrations. Thus, the final concentration of the probe in each well is 1 μM, and the final concentration of the TILs is 2.5×10⁶ cells/ml in 20 μl physiological working solution containing 10 mM phosphate buffer around pH 7.3. Other TILs concentrations can be used, as to ensure two aspects: first, to get a reasonable signal-to-noise ratio due to product accumulation during at least 1 hour, and second, to allow for intercellular interaction. The acidification process is monitored each 5 min during 1 hour of incubation at 37° C. by a commercial fluorescence scanner. First, the scanner monitors the acidification process without sealing in an air-exposed chamber (“OPEN” mode) during 30 min (6 cycles) and then, to avoid ventilation of CO2 and NH3 from each well, the multi-well plate is sealed hermetically (ThermalSeal RT™, EXCEL Scientific, Inc.) (“CLOSE” mode) and measurements are taken in an air-sealed chamber. Next, the acidification process is monitored again during 30 min (6 cycles). In order to increase the signal to noise ratio, the fluorescence intensities at 513 nm is measured sequentially under excitation at 455 nm and 403 nm per well.

In each test, the metabolic activity profiles of TILs is monitored in the basal state and under the influence of the following ten reagents diluted in working solution in 8 different concentrations: PHA (0.4-50 μg/ml), CONA (0.8-100 μg/ml), PMA (0.03-10 μg/ml), LPS (0.03-10 μg/ml), MBP (0.4-50 μg/ml), MelanA (0.08-1 μg/ml), PSA (0.4-50 μg/ml), Glucose (0.4-5 μg/ml), L-glutamine (0.03-4 μg/ml) and Rapamycin (0.6-50 μg/ml).

Other reagents can also be used, for example hormones such as estradiol, cancer specific antigens such as carcinoembryonic antigen (CEA), cytokines and chemokines such as IL2, vitamins, hormones, drugs, antibodies of the immune system, neurotransmitters, different cancer peptides and specific viruses or their fragments such as human papilomavirus (HPV).

The probe used in this test is 8-Hydroxypyrene-1,3,6-trisulfonic acid (HPTS), or other probe that allows sensitive measurements of minor pH changes in the physiological range around pH 7.

The final calibration curve used in the MA test is carried out by pH-glass electrode measurements of sequential titration of WS (containing 2 μM HPTS). The pH measurements and the fluorescence measurements of the titrated samples are carried out at 37° C. The samples are loaded into a multi-well plate and the fluorescence intensities under EX403 nm and EX455 nm are measured at 513 nm using the fluorescence scanner.

For the “OPEN” and “CLOSE” states, a calibration polynomial curve is constructed, allowing to measure pH values and accumulated acidification equivalents as a function of the ratio between the fluorescence intensities measured at 513 nm, under excitations at 403 nm and 455 nm, respectively.

The equations obtained are used for the analysis of TILs (Table 2). X represents fluorescence intensity at Ex. 403 nm/fluorescence intensity at Ex. 455 nm.

TABLE 2
Equations for determining pH and HCl. X represents fluorescence
intensity at Ex. 403 nm/fluorescence intensity at Ex. 455 nm.

| | pH | HCl |
|---|---|---|
| Open | Y = 0.011X⁴ − | Y = 0.006X⁴ + |
| | 0.136X³ + 0.641X² − | 0.129X³ − 0.982X² + |
| | 1.528X + 8.266 | 3.971X − 2.276 |
| Close | Y = 0.013X⁴ − | Y = −0.006X⁴ + |
| | 0.164X³ + 0.747X² − | 0.131X³ − 1.011X² + |
| | 1.723X + 8.415 | 4.168X − 2.581 |

### Data Analysis

Computation, analysis, and data mining are done by using the following Statistical Package; EXCEL 2007, OriginPro 8, SAS Edition 9.2, PASW Modular client 13.0 (formally called Clementine, part of SPSS). Results are expressed as means±standard error of the mean. Statistical significance between healthy and cancer patients for variant models is calculated using chi-square.

First, data is normalized to improve the signal to noise ratio, for example by performing k-means cluster analysis on all observations, and outliers are excluded from further analysis.

Second, a data mining algorithm is constructed based on a set of algorithms from a family of Decision Trees/Rule Induction (C5, CART, CHAID, ASSOCIATION RULE) and log linear model (Logistic Regression). A set from the family of ten different models including data mining, machine learning and statistical modeling are used for example applying SAS 9.3 and Clementine software (V13.0). The graphical method which is based on the cumulative gain charts produce by Clementine software (V 13.0) can be used to evaluate results.

Then the samples are randomly divided into a training set, which may include 70% of the samples, and the remaining 30% of the samples are used to evaluate the classification result.

### Example 7—Metabolic Activity (MA) Test Design and Characteristics

Metabolic activity (MA) tests are performed according to the methods described in U.S. Pat. No. 8,728,758, which is incorporated herein by reference and is summarized below.

Tumor infiltrating lymphocytes (TILs) are obtained as described in the previous examples. For each TIL sample, a 384 multi-well plate is loaded with 20 μl containing physiological working solution at 10 mM buffer around pH 7.3, TILs for example at final concentration of ^{˜}2.5×10⁶ cells/ml, 1 μM pH probe (HPTS), and one of ten stimulating reagents in eight increasing concentrations described in Example 6. The MA test is carried out using a commercial fluorescence scanner. The extracellular acidity kinetic profiles were measured either under air-open (“OPEN”) or hermetically-sealed closed (“CLOSE”) states. The records enable to measure the real-time accumulations of ‘soluble’ versus ‘volatile’ metabolic products (lactic acid versus CO2 and NH3), thereby differentiating between oxidative phosphorylation, anaerobic glycolysis and aerobic glycolysis (“Warburg effect”). The MA rate profiles are calculated and examined to determining the efficiency of TILs for inhibiting tumors.

The non-toxic, membrane-impermeant, ratiometric molecular pH-probe can be, for example, 8-hydroxypyrene-1,3,6-trisulfonic acid (HPTS) with a pKa of ˜7.3 in aqueous physiological buffers. Calibration polynomial curves are constructed for the “OPEN” and “CLOSE” states, allowing to measure pH values and accumulated acidification equivalents as a function of the ratio between the fluorescence intensities measured at 513 nm, under excitations at 403 nm and 455 nm, respectively. The acidification calibration curves are obtained for the working solution (WS) and for the WS diluted 5 times with saline (10 mM and 2 mM phosphate buffer, respectively).

The equations obtained from the final calibration curves are used for quantitative analysis of TILs metabolic activity.

### Example 8—Isolation of TILs from Multiple Tumor Types

### Method

### Pre-REP:

TILs from patient's tumors were isolated and a primary TIL culture was established for each, with the potential to specifically recognize and eliminate malignant cells.

Briefly, a tumor specimen was washed four times by filtering through a steriflip filter tube in PBS supplemented with 50 μg/ml gentamycin. The tumor specimen was then dissected into 1-8 mm3 fragments using a sharp scalpel, and 20-30 fragments were placed in up to five units of G-Rex10 flask in different culture mediums supplemented with 3000 IU/ml IL-2. The G-Rex10 flasks were incubated in a humidified incubator at 37° C. in 5% CO₂ for five days. On day five and every 2 to 3 days after, the media replacement necessity was determined based on glucose and lactate levels. When the glucose level was measured to be below 40 mg/dL or the lactate level was measured to be above 10 mM, the media was refreshed by aspirating one-half of the media from the G-Rex and replacing it with a fresh medium supplemented with IL-2. A pre-REP TILs culture was considered established after 12-18 days when the number of TILs in the culture reached a minimum of 5×10⁶ cells. The produced TILs were then washed and cryopreserved in CryoStore5 preservation solution.

### REP

In the following REP process, the TILs are further expanded to produce billions of cells that can later be re-infused to the patient. The TILs produced in the pre-REP culture were further expanded for 14 days. The medium screening experiments were performed using G-Rex6M well plate (each well has a 10 cm² surface area). The TILs were seeded together with irradiated peripheral blood mononuclear cells (PBMCs), as feeder cells, at a ratio of 1:100 TILs: feeder cells in the 12.5 ml of the tested culture mediums supplemented with 30 ng/ml anti-CD3 antibody (OKT-3) and 3000 IU/ml IL-2. Feeder cells were produced from PBMCs isolated from apheresis and irradiated in 50 Gy. In each experiment, PBMCs from two different donors were pooled and cryopreserved. On the fifth day of the REP culture, 12.5 ml culture medium supplemented with 30 ng/ml OKT-3 and 3000 IU/ml IL-2 were added to the culture. On days 7 and 11 of the REP culture 38 ml of culture medium supplemented with 30 ng/ml OKT-3 and 3000 IU/ml IL-2 were added to the culture.

By the end of the REP process, the cells were washed, counted, their viability was determined using NC200 automated cell counter, and also characterized by the ACEA Biosciences NovoCyte flow cytometer.

### Results

### Pre-REP Cell Yield:

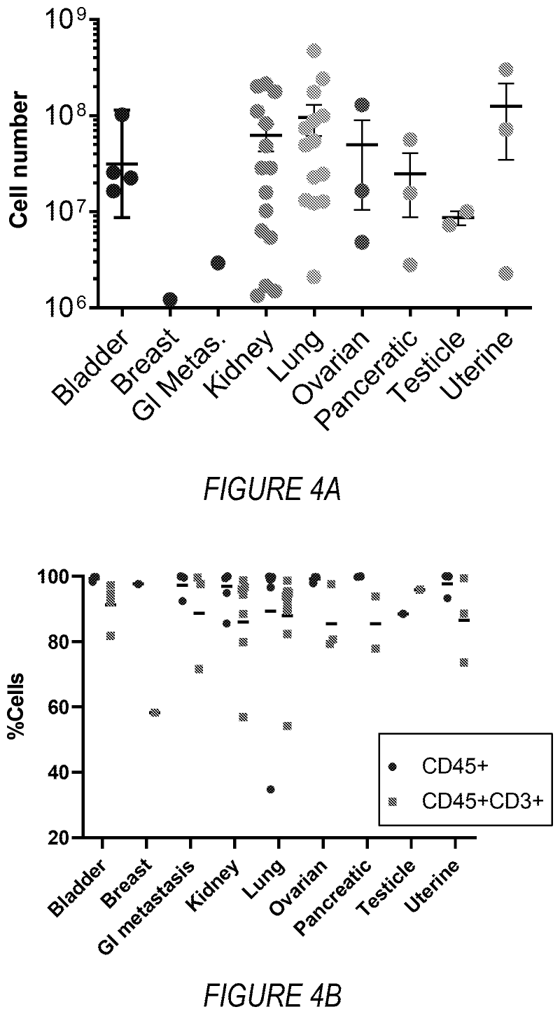

TILs were generated from tumor tissues from bladder, breast, lung, kidney, ovarian, uterine, pancreatic and gastrointestinal cancer and were analyzed by flow cytometry (FIGS. 4A-4B). High T-cell content is shown by the percentage of CD3⁺CD45⁺ T cell within the CD45⁺ cells, examined using a general marker of leukocytes (FIG. 4B).

### Rep Yield:

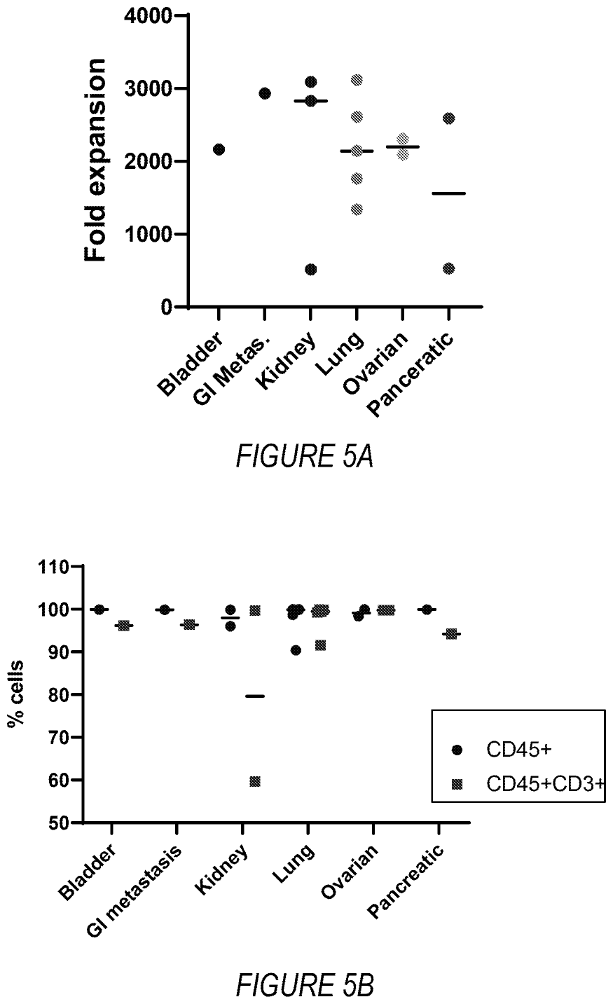

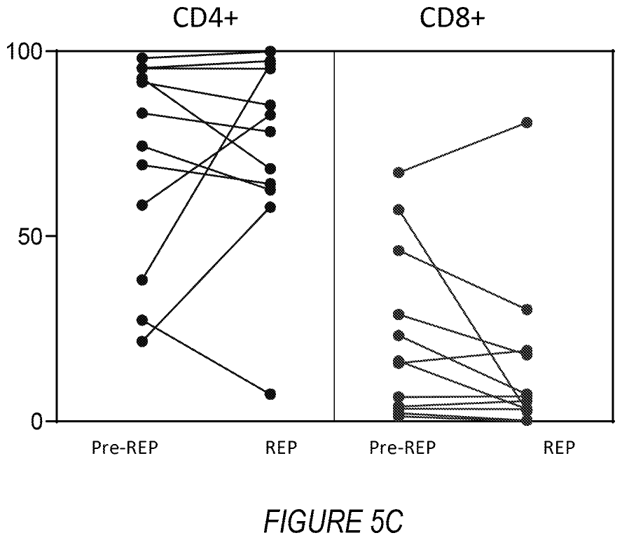

A high fold expansion is observed across the multiple tumors, with an average expansion fold of over 2,000 (FIG. 5A). Flow cytometry analysis of the cells expanded in REP shows a very pure CD45⁺ leukocyte content, of which the vast majority are CD3+ TILs (FIG. 5B). The T-cell CD4+ and CD8+ subpopulations are donor dependent and remain stable between pre-REP and REP (FIG. 5C).

### Example 9—Characterization of TILs from Three Patients' Tumors

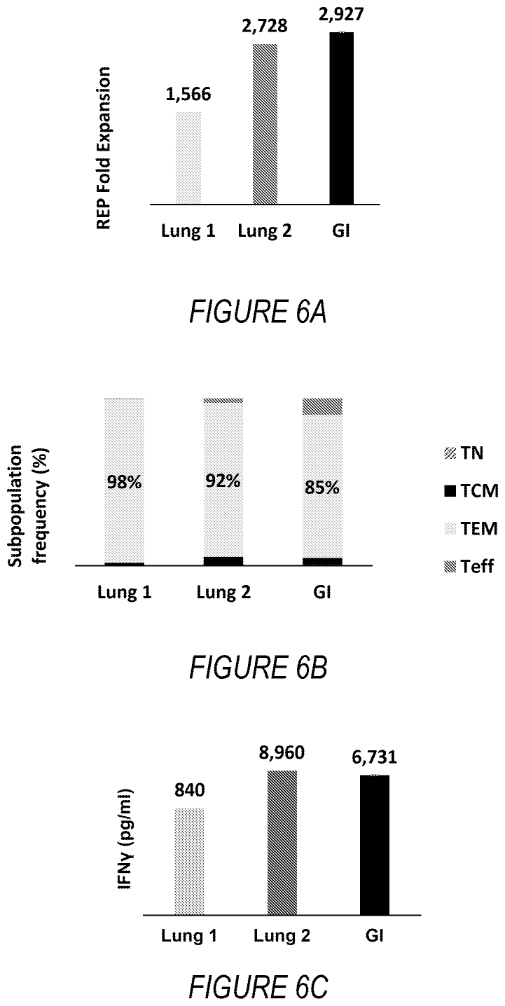

TILs from three patient's tumors, two with lung cancer and one with gastrointestinal (GI) cancer, were isolated and expanded as described in Example 8. All three showed a high expansion rate of over 1000-fold (FIG. 6A).

The frequency of T cell subpopulations was analyzed in the CD45/CD3 population: naïve T cells, central memory T cells, effector memory T cells and T effector cells (FIG. 6B). The memory profile was determined by immunostaining for CD45RO/CCR7. The major population of TILs for all three patients was that of effector memory T cells (98%, 92% and 85%). These cells are capable of preserving a longer immune response compared to terminally differentiated T effector cells.

IFNγ secretion from TILs from the three patients following stimulation with Dynabeads (anti CD3/CD28/CD137) for ˜24 hours, was assessed by ELISA assay (FIG. 6C). All three REP cultures demonstrated high secretion of IFNγ in response to polyclinic activation by beads.

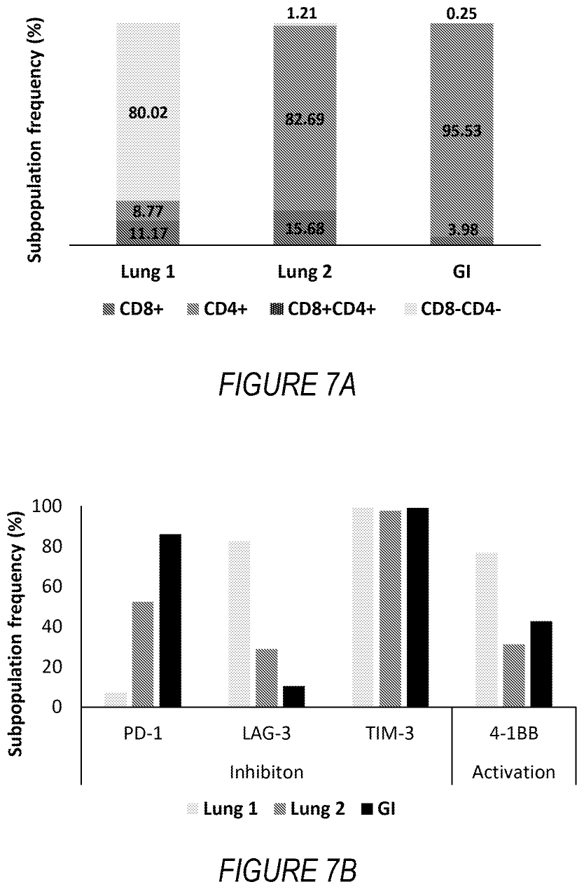

Flow cytometry analysis of the TILs from the three patients shows the percentage of different CD4 and CD8 subpopulations (FIG. 7A). The TILs phenotype shows an inter donor variability (high diversity between patients).

A variable inter-patient expression was observed by flow cytometry analysis of inhibitory and activation markers of the TILs from the three patients, including the inhibitory markers: programmed death receptor 1 (PD-1), T-cell immunoglobulin and mucin domain-containing protein 3 (TIM-3), and lymphocytes activation gene 3 (LAG-3) and the activation marker 4-1BB (CD 137) (FIG. 7B). High expression of inhibitory PD1, TIM-3 and LAG-3 suggests that combination therapy of TILs with antibodies blocking these inhibitory receptors could provide added therapeutic benefit.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A population of tumor infiltrating lymphocytes (TILs) produced by a method comprising:
a. extracting a biopsy comprising a tumor and/or its surroundings;
b. isolating, culturing, and expanding TILs isolated from said biopsy;
c. determining the metabolic activity (MA) in said TILs by a method comprising measuring in the extracellular environment of the cell the time-dependent acidification profiles due to secretion of:
(i) non-volatile soluble metabolic products and volatile soluble metabolic products;
(ii) non-volatile soluble metabolic products; and
(iii) volatile soluble metabolic products;
wherein said measuring acidification profile of (ii) is affected in an air-exposed chamber, and
wherein said measuring acidification profile of (i) is affected in an air-sealed chamber, and
wherein said measuring acidification profile of (iii) is affected by subtracting the acidification profile of (ii) from the acidification profile of (i), and
wherein all of said time dependent acidification profiles are indicative of the metabolic activity of the cell; and
d. harvesting TILs.

2. The population of TILs of claim 1, wherein said TILs are harvested when the MA is above a predetermined threshold.

3. The population of TILs of claim 1, wherein the method further comprises:
(A) contacting said TILs from step (b) with one or more stimulants prior to, or concomitant with determining the metabolic activity; or
(B) prior to step (d), selecting and expanding TILs having enhanced potency for cell therapy based on said metabolic activity; or
(C) both (A) and (B).

4. The population of TILs of claim 3, wherein said one or more stimulants comprise a synthetic peptide or a cancer-specific peptide.

5. (canceled)

6. The population of TILs of claim 3, wherein said one or more stimulants are selected from the group consisting of NY-ESO-1, Her-2a, ConA, PHA, MAGE-A3 and glucose.

7. (canceled)

8. (canceled)

9. The population of TILs of claim 1, wherein said TILs comprise T cells, B cells, natural killer (NK) cells, macrophages, neutrophils, dendritic cells, mast cells, eosinophils, basophils, plasma cells, antigen presenting cells (APCs), CD4+, CD8+, CD163+, CD20+, CD3+, CD138+, CD163+, CD56+, CD28+, CD69+, FoxP3+, DC-LAMP+ cells, or any combination thereof.

10. A method for producing a population of tumor infiltrating lymphocytes (TILs), said method comprising:
a. extracting a biopsy comprising a tumor and/or its surroundings;
b. isolating, culturing, and expanding TILs isolated from said biopsy;
c. determining the metabolic activity (MA) in said TILs by a method comprising measuring in the extracellular environment of the cell the time-dependent acidification profiles due to secretion of:
(i) non-volatile soluble metabolic products and volatile soluble metabolic products;
(ii) non-volatile soluble metabolic products; and
(iii) volatile soluble metabolic products;
wherein said measuring acidification profile of (ii) is affected in an air-exposed chamber, and
wherein said measuring acidification profile of (i) is affected in an air-sealed chamber, and
wherein said measuring acidification profile of (iii) is affected by subtracting the acidification profile of (ii) from the acidification profile of (i), and
wherein all of said time dependent acidification profiles are indicative of the metabolic activity of the cell; and
d. harvesting TILs.

11. The method of claim 10, wherein said TILs are harvested when the MA is above a predetermined threshold.

12. The method of claim 10, further comprising:
(A) contacting said TILs from step (b) with one or more stimulants prior to, or concomitant with determining the metabolic activity; or
(B) prior to step (d), selecting and expanding TILs having enhanced potency for cell therapy based on said metabolic activity; or
(C) both (A) and (B).

13. The method of claim 12, wherein said one or more stimulants comprise a synthetic peptide or a cancer-specific peptide.

14. (canceled)

15. The method of claim 12, wherein said one or more stimulants are selected from the group consisting of NY-ESO-1, Her-2a, ConA, PHA, MAGE-A3 and glucose.

16. (canceled)

17. The method of claim 10, wherein said tumor is selected from melanoma, non-Hodgkin's lymphoma, Hodgkin's disease, leukemia, plasmocytoma, sarcoma, glioma, thymoma, breast cancer, prostate cancer, colo-rectal cancer, kidney cancer, renal cell carcinoma, uterine cancer, pancreatic cancer, esophageal cancer, brain cancer, lung cancer, ovarian cancer, cervical cancer, testicular cancer, gastric cancer, esophageal cancer, multiple myeloma, hepatoma, acute lymphoblastic leukemia (ALL), acute myelogenous leukemia (AML), chronic myelogenous leukemia (CML), and chronic lymphocytic leukemia (CLL), or any combination thereof.

18. The method of claim 10, wherein said TILs comprise T cells, B cells, natural killer (NK) cells, macrophages, neutrophils, dendritic cells, mast cells, eosinophils, basophils, plasma cells, antigen presenting cells (APCs), CD4+, CD8+, CD163+, CD20+, CD3+, CD138+, CD163+, CD56+, CD28+, CD69+, FoxP3+, DC-LAMP+ cells, or any combination thereof.

19. A method for treating a tumor in a subject in need thereof, said treating comprising administering a population of tumor infiltrating lymphocytes (TILs) produced by a method comprising:
a. extracting a biopsy comprising a tumor and/or its surroundings;
b. isolating, culturing, and expanding TILs isolated from said biopsy;
c. determining the metabolic activity (MA) in said TILs by a method comprising measuring in the extracellular environment of the cell the time-dependent acidification profiles due to secretion of:
(i) non-volatile soluble metabolic products and volatile soluble metabolic products;
(ii) non-volatile soluble metabolic products; and
(iii) volatile soluble metabolic products;
wherein said measuring acidification profile of (ii) is affected in an air-exposed chamber, and
wherein said measuring acidification profile of (i) is affected in an air-sealed chamber, and
wherein said measuring acidification profile of (iii) is affected by subtracting the acidification profile of (ii) from the acidification profile of (i), and
wherein all of said time dependent acidification profiles are indicative of the metabolic activity of the cell;
d. harvesting TILs from step (c); and
e. administering the subject with the TILs from step (d).

20. The method of claim 19, wherein said TILs are harvested when the MA is above a predetermined threshold.

21. The method of claim 19, further comprising:
(A) contacting said TILs from step (b) with one or more stimulants prior to, or concomitant with determining the metabolic activity; or
(B) prior to step (d), selecting and expanding TILs having enhanced potency for cell therapy based on said metabolic activity; or
(C) both (A) and (B).

22. The method of claim 21, wherein said one or more stimulants comprise a synthetic peptide or a cancer-specific peptide.

23. (canceled)

24. The method of claim 21, wherein said one or more stimulants are selected from the group consisting of NY-ESO-1, Her-2a, ConA, PHA, MAGE-A3 and glucose.

25. (canceled)

26. The method of claim 19, wherein said tumor is selected from melanoma, non-Hodgkin's lymphoma, Hodgkin's disease, leukemia, plasmocytoma, sarcoma, glioma, thymoma, breast cancer, prostate cancer, colo-rectal cancer, kidney cancer, renal cell carcinoma, uterine cancer, pancreatic cancer, esophageal cancer, brain cancer, lung cancer, ovarian cancer, cervical cancer, testicular cancer, gastric cancer, esophageal cancer, multiple myeloma, hepatoma, acute lymphoblastic leukemia (ALL), acute myelogenous leukemia (AML), chronic myelogenous leukemia (CML), and chronic lymphocytic leukemia (CLL), or any combination thereof.

27. The method of claim 19, wherein said TILs comprise T cells, B cells, natural killer (NK) cells, macrophages, neutrophils, dendritic cells, mast cells, eosinophils, basophils, plasma cells, antigen presenting cells (APCs), CD4+, CD8+, CD163+, CD20+, CD3+, CD138+, CD163+, CD56+, CD28+, CD69+, FoxP3+, DC-LAMP+ cells, or any combination thereof.

28-31. (canceled)
